(19) United States
(12) Patent Application Publication
(10) Pub. No.: US 2007/0293947 A1
(43) Pub. Date: Dec. 20, 2007

(54) Multi-part implants for combined repair of hyaline and meniscal cartilage in joints

(76) Inventor: Kevin A. Mansmann, Paoli, PA (US)

Correspondence Address: Patrick D. Kelly;Attorney for Applicant, 11939 Manchester, #403, St. Louis, MO, 63131, US

(21) Appl. No.: 11/471,090

(22) Filed: Jun. 19, 2006

(51) Int. Cl. A61F 2/30; A61F 2/38; A61B 17/16

(52) U.S. Cl. 623/14.12; 623/20.16; 606/79

(57) ABSTRACT

Surgical implants use combined anchoring components to replace meniscal or labral cartilage, in ways that provide strong reinforcement while emulating natural anchoring. An arc-shaped polymer segment is coupled to an anchoring rim made of shape-memory material, which will fit into a groove prepared in a bone surface using specialized tools. A fabric material or anchoring ring is provided above the polymer segment, and can be secured to a knee capsule or other soft tissue. Fabric strips can extend out from the tips of the polymer arc, for additional anchoring. An additional polymer segment can also be provided to replace a hyaline cartilage layer, with a porous bottom surface to promote tissue ingrowth. By using peripheral rather than central anchoring, such implants can be given very high strength and stbility, to last for multiple decades.

### FIELD OF THE INVENTION

This invention is in the field of surgery, and relates to surgical implants for repairing or replacing cartilage in knee or shoulder joints.

### BACKGROUND OF THE INVENTION

Background information on surgical implants that can be used to replace damaged cartilage, in articulating joints such as knees or shoulders, is available in various books, patents, and articles that are cited and discussed in several prior patent applications by the inventor herein, an orthopedic surgeon. Those applications include several applications published under the Patent Cooperation Treaty (PCT) system (including PCT applications WO 03/103543, WO 03/103543, and WO 05/032426), and on the U.S. patent and Trademark Office website (including published applications 2005/0287187 (Hydrogel implants for replacing hyaline cartilage, with charged surfaces and improved anchoring), 2004/0133275 (Implants for replacing cartilage, with negatively-charged hydrogel surfaces and flexible matrix reinforcement), and 2002/0173855 (Cartilage repair implant with soft bearing surface and flexible anchoring device), and issued U.S. Pat. No. 6,629,997 (Meniscus-type implant with hydrogel surface reinforced by three-dimensional mesh). The contents of those published items are incorporated herein by reference, as though fully set forth herein.

Two different and distinct classes of cartilage are of interest herein, and both types are present in knee, hip, shoulder, and wrist joints. Those two categories are referred to herein as hyaline cartilage and meniscal cartilage. The difference between them is crucial, since it leads to very different types of reinforcing and anchoring structures.

As used herein, hyaline cartilage includes any natural cartilage that is directly affixed and anchored to a bone surface (these bone surfaces are often called condyles; however, that term is not always used consistently, since some people limit it to rounded ends on elongated bones, while others also use it to refer to the rounded socket surfaces in hip and shoulder joints). Hyaline cartilage is relatively thin, which effectively coats and covers a bone surface. The side which is anchored directly to a bone surface can be called the base, under, inner, interior, or anchoring side, or similar terms), while the other side (which can be called the exposed, outer, lubricated, or articulating side or surface, or similar terms) has a smooth and lubricated surface that enables non-abrasive sliding and “articulation” of the cartilage surface against a similar smooth and lubricated surface of a different cartilage segment, in a joint. In a knee, the femoral runners (which cover the two parallel rounded surfaces at the bottom of a femur or thigh bone), the tibial plateau (which covers the roughly horizontal surface at the top end of a tibia or shin bone), and the patellar cartilage (on the back side of the kneecap) are all hyaline cartilage segments.

Because hyaline cartilage is a water-filled hydrogel that cannot be as hard as bone, it is present only in relatively thin layers, to minimize the risks of distortion, shearing stresses, and other problems that could lead to tearing and damage. The hydrogel structure is created and reinforced mainly by collagen, the fibrous protein that holds together nearly all types of soft tissues in animals. Within the hydrogel, a structural transition layer called the “subchondral zone” helps anchor the cartilage to the supporting hard bone material. That transitional zone is traversed by thousands of collagen fibers that extend downward into the underlying hard bone, and upward into the cartilage layer, thereby creating a gradual and reinforced transition rather than an abrupt discontinuity between hard bone and soft cartilage.

By contrast, meniscal cartilage segments in a knee are not directly affixed or anchored to bone surfaces. Instead, they have shapes that are generally comparable to a segment from an orange or other citrus fruit, but with a larger curvature (or “arc”) that more closely resembles the letter “c”. This shape is also sometimes referred to, especially in older texts, as a “semilunar” shape, since it somewhat resembles the moon, midway between the crescent and half-moon stages.

Each knee joint in a human contains two segments of meniscal tissue. A lateral (or external) meniscus is on the outer side of each knee, and a medial (or internal) meniscus is on the inner side of each knee. This is consistent with how the knee is regarded as a “two compartment” assembly, by arthroscopic surgeons. The patella (kneecap) and the anterior cruciate ligament (abbreviated as ACL) occupy and effectively block the center of any frontal approach pathway, in a manner that prevents a “centerline” approach. Therefore, even if an entire tibial surface and both meniscal segments need to be replaced in a damaged knee in a patient, the preferred approach for arthroscopic repair is usually to repair either the medial or lateral “compartment” first, and then repair the second compartment after the first compartment has been essentially completed. Accordingly, the implant devices described and illustrated herein are designed for “unicompartmental” repair of either the medial or the lateral side of a knee (those two compartments are nearly symmetric with each other). If a complete tibial surface and both meniscal segments are to be replaced by such implants, the surgeon will insert one implant on one side of the knee, and will then insert a second implant on the other side of the knee, with two different insertion pathways that flank the kneecap and ACL to leave them intact and undamaged.

On either the medial or lateral side of a knee, the thickest region of the meniscus is around the periphery, which also can be called the circumference, rim, or similar terms. This outer rim is anchored to the inside of a fibrous “capsule” made of tendons, ligaments, and other soft tissues. The capsule encloses the knee joint and holds in synovial fluid, the slippery liquid that lubricates the joint.

The two ends of each wedge-shaped meniscal arc (also called the “arcuate tips”) are coupled, via ligaments, to bony protrusions that rise above the center of the roughly horizontal “plateau” on the upper end of a tibia bone. Those bony protrusions usually are called the tibial “spine”. As a knee is bent or extended, they slide within the groove between the two rounded and parallel runners on the bottom of the femur (thigh) bone, in a manner that helps stabilize the knee.

The inner edge of a meniscus (i.e., the thinnest portion of the wedge-shaped segment) is often called the apex, margin, or similar terms. It is not anchored; instead, as a person walks or runs, each meniscus is somewhat free to move, as it is squeezed between a femoral runner above it, and a tibial plateau beneath it. Accordingly, as mentioned above, each meniscus must have not just one but two slippery and lubricated surfaces.

The bottom lubricated surface of each meniscus is relatively flat. As described above, instead of being anchored directly to tibial bone, it moves and slides (in a constrained manner, with only a short travel path) on top of the hyaline cartilage on the tibial plateau, in a knee.

The upper surface of a meniscus is also smooth, wet, and slippery. It is sloped and rounded, in a manner that forms a rounded, concave, bowl-shaped surface on the top of the tibial plateau, as illustrated (in a somewhat exaggerated manner) in FIG. 1. This enables the combined tibial plateau and meniscal arc to closely conform to the rounded convex shape of a femoral runner, throughout a wide range of motion in the knee joint. The bowl-shaped supporting surface, which is provided by the tibial plateau combined with two meniscal segments, provides the two femoral runner with much better reinforcement, stability, and support (including lateral support) than could be provided by a relatively flat surface of the tibial plateau alone. As a result, meniscal segments are essential to proper functioning of knee joints; if either meniscus is surgically removed after an injury, the knee will begin to develop serious arthritis within a few months or years.

Accordingly, meniscal cartilage (i.e., wedge-shaped cross-sections, with two lubricated surfaces) is quite different from hyaline cartilage (i.e., thin surface layers anchored directly to bone, with only one sliding surface). Indeed, because of their different sizes, shapes, and anchoring modes, these two classes of cartilage are even made from different types of cartilage. Meniscal cartilage is made from specialized “fibrocartilage”, which is heavily reinforced by long collagen fibers that are oriented along the lengths of the arcs. These long fibers emerge from the tips of the meniscal arcs, and merge with ligaments that affix the tips of the meniscal to the bony protrusions of the tibial spine. By contrast, hyaline cartilage has much shorter structural and reinforcing fibers, and as a result, it is not as strong or tough as fibrocartilage.

Because meniscal segments in human knees are subjected to frequent combinations of compressive and tensile stresses (and sometimes abrasion, especially in people suffering from chondromalacia, arthritis, injuries, or other problems that can cause a loss of smoothness in cartilage surfaces), meniscal damage often occurs in humans, and occasionally in livestock and other animals. Therefore, various efforts have been made to provide surgical implants that can be used to replace damaged meniscal segments, in knees. However, because of their complex structures and anchoring, and because of the need to create and sustain very smooth and constantly wet surfaces on both the upper and lower surfaces of each meniscal wedge, meniscal implants in the prior art have not been entirely adequate.

In the relevant art, a number of patents have disclosed various types of proposed implant devices for replacing damaged meniscal segments. For example, U.S. Pat. No. 4,502,161 (Wall 1985) describes a resilient wedge-shaped segment, affixed to a metal bracket that will be screwed into the side of the shaft of the tibia. That device has some utility; however, because its anchoring structure does not resemble or emulate the anchoring system of a healthy native meniscal segment, that type of implant could not move, respond, and behave in the same ways that a natural meniscal segment will move and behave.

U.S. Pat. No. 4,919,667 (Richmond 1990, assigned to the Stryker Corporation, a major supplier of orthopedic implants) described a laminated wedge-shaped multi-layer device, with its ends (tips) affixed to steel or similar pins that will be driven into the tibial bone at appropriate locations, and with felt layers inside the wedge, to provide the wedge with a resiliency that resembles natural cartilage. That may have offered some improvement over prior designs, but it still fell short of being optimal. In particular, the '667 patent did not address the crucial issue of how the anchoring pins that were described, for anchoring that type of implant, apparently would need to penetrate and damage the natural cartilage (or a cartilage-replacing implant) that sits on top of the tibial plateau. That is a major issue, because the act of driving steel pins through the smooth-surfaced cartilage on a tibial plateau would pose grave risks of damaging that cartilage surface, and creating an abrasive surface that would also damage the femoral runner as well. However, that apparent problem was not addressed in the Richmond '667 patent.

U.S. Pat. No. 5,735,903 (Li et al 1998) described a resorbable meniscal implant designed to hold “meniscal fibrochondrocyte cells”, which hopefully would be able to regenerate actual biological cartilage, during a span of months after implantation. However, those and numerous other efforts to replace cartilage in load-bearing joints, by using transplanted cells to generate new biological cartilage, have generally failed to overcome the problems that arise when a resorbable implant begins to release particles and debris into a repaired joint. The release of particles and debris will begin to occur at some point during the gradual digestion and deterioration of a resorbable implant, when the implant reaches a state of being mostly but not entirely dissolved and digested by enzymes and body fluids. Because of that problem, the use of resorbable implants that hold transplanted cells, for regenerating new cartilage, has been limited to only two relatively small niches: (i) cosmetic repair of cartilage in body parts that are not subjected to loadings and stresses, such as ears and noses; and, (ii) repair of small joint defects caused by injuries, usually limited to relatively young patients. Those niches do not apply to meniscal segments that need to be replaced. Since the crucial problem of debris being released by partially-dissolved resorbable implants has not been overcome (and likely cannot be overcome, because of the inherent nature of how resorbable implants are digested and dissolved, over a span of time), there appears to be little or no serious interest in trying to use resorbable materials carrying transplanted cells, for meniscal replacements.

U.S. Pat. No. 6,629,997 (by Mansmann, the same inventor herein) took a different approach, and disclosed a meniscal implant made of a synthetic polymer that forms a flexible hydrogel when saturated with water. The hydrogel polymer enables the gel-like material to behave in ways similar to natural cartilage, and it provides smooth wet surfaces on both the top and bottom sides of the wedge. A fibrous reinforcing mesh is embedded within the polymer; however, it does not reach or disrupt the smooth lubricated surfaces of the hydrogel material, and instead is exposed around the periphery of the flexible implant, in a manner that allows the implant to be anchored to the tendons and other tissues that form the capsule around the knee. The reinforcing mesh can also enable the tips of a meniscal implant to be secured to the tibial bone.

Mansmann's approach was disclosed to the major companies that make orthopedic implants, and it was subsequently followed and expanded upon, in U.S. Pat. No. 6,994,730 (Posner 2006, initially assigned to Howmedica Osteonics Corp., which was subsequently purchased by the Stryker Corporation). Posner '730 disclosed a multi-component implant for knee repair, containing (i) a tibial component that will be affixed to a prepared tibial bone surface; (ii) a femoral component that will be affixed to a prepared femoral bone surface; and, (iii) a meniscal component, which will be affixed to soft tissue in the joint being repaired. The recommended method for anchoring the wedge-shaped meniscal component of such an implant is by suturing it to a carved-out peripheral remnant, from the original meniscal wedge. That approach purportedly could avoid the need for creating a new set of attachments that would affix the tips of the wedge-shaped implant, to the tibial spine protrusions.

However, the Posner '730 patent appears to have taken a flawed approach, which is likely to jeopardize and diminish the strength, stability, and long-term durability of the implants disclosed therein. This problem arises from Posner's efforts and intent to leave behind a peripheral remnant from the outer edge of a natural meniscal wedge that will be partially removed and replaced. To enable and facilitate that approach, the Posner '730 patent directly and repeatedly states that the tibial implant should be anchored to “a central portion” of the tibial surface that is being repaired.

The term ”central portion”, as used in the Posner '730 patent, refers to either a medial or lateral segment of a tibial plateau; the text and drawings make it clear that those references to “a central portion” do not mean that the tibial spine, in the true center of the tibial plateau, should be removed. Instead, according to Posner '730, either a central portion of a lateral tibial compartment, or a central portion of a medial tibial compartment, should have the damaged cartilage removed, and the underlying bone should be “resected”, by drilling and/or machining out a non-circular portion of the bone (such as by creating an I-shaped resection, as illustrated in FIG. 10 of Posner '730). That type of bone resection can be performed in a predetermined pattern by using positioning pins, cutting templates, and cutting and grinding tools, as illustrated in FIGS. 3-9 of the Posner '730 patent. Subsequently, when the I-shaped anchoring component on the bottom of a tibial implant is emplaced in the resected bone surface, the accommodating non-circular shapes of the bone surface and the implant can prevent the implant from rotating or becoming loosened or dislodged, inside the knee joint.

The problem and risk that Posner and Howmedica apparently failed to recognize and address can be summarized as follows: a implant that is anchored only to the center portion of a bone surface will not and cannot be as strong, stable, or durable (over a span of decades) as an implant that is anchored to a bone surface, around the periphery of the implant base.

This result arises from mechanical factors that are often referred to as “bending moments”, rotational (or torsional) vectors, or similar terms. It can be understood by considering the design of steering wheels, in cars and trucks. If a driver attempted to drive a car by gripping a rotatable shaft, having a diameter of only about four to six inches, the driver would not have firm and reliable control over the steering of the car. Therefore, steering wheels are substantially enlarged, typically to dimensions of about 14 to 15 inches (or even larger, in large trucks), and drivers are told to grip a steering wheel with both hands, on opposing sides of the wheel, to provide better control over a car they're driving. If a two-handed grip is properly distributed (i.e., if the hands are placed more than a foot apart, on the steering wheel), they will provide optimal control over the car, for the driver. Even if only a single hand is used by a driver, the act of placing the hand on an enlarged wheel, at a location that is a substantial distance from the center of the wheel, will still provide substantially better and stronger control over the wheel than could be obtained by gripping only the shaft or axle itself.

Other examples can be provided by various tools, and by everyday experience. As two brief examples, a long-handled wrench can be used to exert much more torque on the head of a bolt than a short-handled wrench; and, a screwdriver with a large handle can be used to exert much more torque on a screw, than a screwdriver with only a slim and narrow handle.

Beyond that, there is also another factor that is crucial to the long-term durability of implants in knees or other joints. Most implants typically do not fail somewhere in the middle, as one of the earliest triggering events; instead, they tend to be pushed or pried loose, somewhere around the periphery, as one of the earliest events that will trigger a series of subsequent problems that eventually culminate in failure. Therefore, if the periphery of the implant is strongly anchored and secured, in a way that prevents any part of the periphery from being pushed around, pried up, or otherwise distorted, the risks of eventual failure can be substantially reduced, when compared to an implant that is anchored only in its center while its periphery is not firmly secured.

These factors become crucially important in cartilage-replacing implants for knees, since such implants should be and must be designed to last not just for years, but for decades. Any non-resorbable implant for repairing a knee in a grown patient should be designed to last for the entire remaining life of the patient, no matter how long the patient may live after the surgery. That is true for patients in their forties or fifties, or even in their twenties or thirties, and it applies even to a teenager who suffered an injury while playing sports, where the desired life of the implant may be seventy years or even longer. Regardless of a patient's age or condition, any design modifications that can make a knee repair implant stronger, more stable, and more durable, are beneficial and desirable.

In addition to meniscal segments in knees, thickened wedge-shaped cartilage segments also are present in shoulder, hip, and wrist joints. These cartilage segments are usually called “labrum” or “labral” segments, since they are roughly lip-shaped (the root word labium is the Latin word for “lip”). In the hips and shoulders, the labral segments form what are, in effect, enlarged rims around the outer edges of the concave surfaces that cover the socket portions of the large ball-and-socket joints. However, because of the inherent weakness of hyaline cartilage (with its short reinforcing fibers made of collagen), the thicker and therefore more vulnerable labral rims which form the edges of the cartilage socket segments are made of the same type of more heavily reinforced fibrocartilage that is used to make meniscal segments, in knees.

Accordingly, because labral cartilage shares several crucial traits with meniscal cartilage, the words “meniscus” and “meniscal” are used broadly herein, to also include and apply to labral cartilage. As will be recognized by orthopedic surgeons and researchers, the teachings herein concerning implant segments for replacing meniscal cartilage segments, in knees, also can be adapted to design and provide implants for replacing labral cartilage segments, in other joints.

Accordingly, one object of this invention is to disclose multi-component implants for knee repairs which contain or enable meniscal replacement components, and which have improved anchoring means that provide higher levels of strength, stability, and durability than previous meniscal implants in the prior art.

Another object of this invention is to disclose a class of implants for meniscal repairs that can utilize enhanced types of anchoring structures that were initially developed for other types of cartilage repair.

Another object of this invention is to disclose a class of implants for meniscal repair implants that contain a combination of various traits and components, including improved anchoring means combined with features such as chemically-imparted negative electrical charges on their articulating surfaces that will render those surfaces better able to interact in desired ways with the lubricating components of synovial fluid.

These and other objects of the invention will become more apparent through the following summary, drawings, and detailed description.

### SUMMARY OF THE INVENTION

Devices, methods, and tools are disclosed for surgical implants that use a combination of anchoring components to replace meniscal cartilage segments in knees, in ways that provide strong reinforcement while also emulating the anchoring of natural meniscal segments. Such anchoring components include: (1) an anchoring rim made of a nitinol alloy or other shape-memory material that can be manipulated by squeezing, chilling, or other means, to minimize tissue damage during insertion; (2) anchoring receptacles that are anchored in hard bone before an implant is inserted into a knee joint, while room to work is available; (3) anchoring pegs on the anchoring rim, which will lock in place when pushed into the anchoring receptacles; (4) a polymer insert gripped by the anchoring rim, which will anchor a flexible reinforcing fabric that is also embedded in an arc-shaped polymer segment that will replace a meniscal wedge; (5) a flexible fabric extending above the upper edge of the meniscal polymer segment, which can be sutured or stapled to the knee capsule or a remnant of a meniscal rim; and, (6) fibrous material that extends out of the tips of the meniscal polymer segment, which can be anchored to ligament or meniscal remnants affixed to the tibial spine. All of these components can be provided in a single implant that can be arthroscopically inserted. The implant can also provide a polymer layer that will replace hyaline cartilage on a tibial plateau, with a porous bottom surface that will promote tissue ingrowth for even stronger anchoring.

Arthroscopic tools are also disclosed for preparing a tibial bone to receive and support such an implant. One set of tools will create a smooth planed surface from which natural cartilage has been removed, while another set of tools will create a groove around the periphery of the surface, to help hold and reinforce the anchoring rim of an implant.

### BRIEF DESCRIPTION OF THE DRAWINGS

FIG. 1 is a perspective cutaway view showing an implant for replacing cartilage in a knee, with (i) an oval-shaped anchoring rim made of a shape-memory alloy; (ii) a flexible and resilient layer that spans the anchoring rim, with a smooth and wet upper surface, and a porous bottom layer for promoting tissue ingrowth; (iii) a wedge-shaped component that is sized and shaped to replace meniscal cartilage; and, (iv) fibrous reinforcing material that attaches the meniscal wedge to the anchoring rim, and that extends above the meniscal wedge to provide a peripheral anchor that will be sutured or stapled to the capsule that surrounds a knee joint.

FIG. 2 is a cutaway view of a modified knee implant that also contains two hoops made of shape-memory material that will gently press outwardly against the inside of the knee capsule, with both hoops affixed to a reinforcing layer embedded within the flexible wedge that will replace meniscal cartilage.

FIG. 3 is a cutaway view of an implant having a shallower wedge-shaped polymer component, with no sliding interface between the hyaline-replacing layer, and the meniscal or labral wedge. This design can be used for (i) replacing a labral segment in a hip or shoulder, or (ii) affixing an “arcuate tip” of a meniscal segment, to a hyaline-replacing layer. The tip of the wedge segment has a flexible fabric emerging from, it for anchoring purposes, and the anchoring rim shows a shape-memory alloy ring, embedded within a polymer.

FIG. 4 is a perspective view of a planing tool with a rotating drive shaft inside a non-rotating sheath or handle, for arthroscopic smoothing of a relatively flat bone surface such as a tibial plateau.

FIG. 5 is an enlarged perspective view of the underside of a planing tool, with a grinding burr mounted on the end of a linear drive shaft that enters one end of a cowl, and with four adjustable feet surrounding the exposed burr.

FIG. 6 is a perspective view of the closed covering surface of the cowl of a planing tool.

FIG. 7 is a perspective view of a planing tool for a femoral runner or other curved surface, with a handle or sheath that encloses a rotating shaft, entering the cowl at a right angle relative to the axis of the grinding burr, using a mechanism that allows the cowl to rotate with respect to the handle or sheath.

FIG. 7 is a perspective view of a femoral planing tool, with the handle and drive shaft perpendicular to the axle of the grinding burr, using a mechanism that allows the cowl to rotate with respect to the handle and drive shaft.

FIG. 8 is a perspective view of the underside of a femoral planing tool, showing the burr, the underside of the cowl, and four adjustable “feet” surrounding the burr.

FIG. 9 depicts a planing tool pressed against a femoral runner in a knee joint, to indicate the size of the planing tool. The bone depiction is stylized; during an operation, it will be surrounded by skin and other tissues.

FIG. 10 is a cutaway view of planing tool for a femoral runner, indicating how a gear surface on the drive shaft can drive the rotation of the burr.

FIG. 11 depicts a different drive mechanism for a femoral planing tool, using a flexible drive belt that passes through an access tube.

FIG. 12 is a perspective view of the bottom surface of a femoral planing tool that uses a drive belt, showing the belt (in tension) passing across a knurled, grooved, or other non-smooth surface on the axle of the grinding burr.

FIG. 13 is a perspective view of a femoral planing tool with a two-part cowl, which allows the top and bottom portions of the cowl to rotate, relative to each other.

FIG. 14 is a side view of a planing tool, showing the “foot” components at an extension that causes them to prepare a flat surface. If the feet are extended farther (lower), the burr can create an arc with controlled curvature, in a bone such as a femoral runner.

FIG. 15 is a side elevation view of a planing tool that utilizes a flat grinding surface at the end of a rotating burr, rather than a cylindrical grinding surface on the side of a burr.

FIG. 16 is a side (elevation) view of a grooving tool, showing a grinding burr, cowl, sheath, and drive interface.

FIG. 17 is an enlarged side view of the burr and cowl of a grooving tool, with a partial cutaway showing the rotating drive shaft inside the nonrotating sheath.

FIG. 18 is a cutaway perspective view of a grooving tool, showing a generally spherical burr at the end of a hollow rotating shaft, the sheath that surrounds the rotating shaft, and a movable cowl affixed to the end of the sheath.

FIG. 19 is a side view of a grooving tool pressed against a flat bone surface, indicating how the cowl can travel through an arc that allows its flat surface to stay aligned with a bone surface, with the rotating spherical burr remaining at a constant depth (or extension) while the handle moves through various angles relative to the flat surface.

FIG. 20 is a top (plan) view of a grooving tool, depicting the slot on the top of the cowl, and an inlet on the sheath that will allow blood, saline solution, and bone chips to be suctioned out of the operating field, and removed via the hollow drive shaft.

FIG. 21 depicts the mounting of a movable cowl at the end of a sheath, showing the cowl mounted on a U-shaped lip at the end of the sheath.

FIG. 22 depicts the interior (bowl) surface of the cowl, showing an indentation that allows the cowl to slide through an arc while interacting with the U-shaped lip at the end of a handle sheath.

FIG. 23 depicts a grooving tool making a round or oval groove on the surface of a bone or other grindable material, guided by a template.

### DETAILED DESCRIPTION

As summarized above, this invention discloses new designs for flexible surgical implants that can be used for arthroscopic repair of damaged meniscal cartilage in knee joints, or damaged labral cartilage in shoulders, hips, or wrists. In any of several preferred embodiments, a single implant device can replace both (i) a relatively thin layer of hyaline cartilage, such as the cartilage that covers the upper end of a tibial bone; and, (ii) a wedge-shaped meniscal or labral segment. In addition, these devices can provide substantially improved anchoring and performance of the meniscal or labral components, compared to any previously known prosthetic implants.

To enable insertion on either side of a patella (kneecap) and anterior cruciate ligament (ACL), a preferred type of knee implant will be sized for “unicompartmental” repair of either the medial (inside) or the lateral (outside) portion of a knee. If both compartments need to be repaired, two such implants will be used, one on each side.

The use of the term “sized”, to refer to various implant components, will be readily understood by orthopedic surgeons and researchers. Briefly, the term “sized” is a shorthand reference which covers, includes, and incorporates all physical dimensions (including “cross-sectional” as well as “overall” dimensions) that will render a sterile polymeric device inside a sealed sterile package actually useful and practical for surgically replacing injured or diseased cartilage in a mammalian joint, as described herein. The term “sized” also avoids unrecognized, unintentional, and unrelated prior art, since the process of “sizing” an implant in a way that causes it to be useful and practical, for surgical implantation as described herein, is not accidental, and instead requires extraordinarily high levels of effort and attention, to obtain the governmental approvals that are required before such devices can be used for medical or veterinary purposes. The necessary government approvals require that such devices must be precision-manufactured, using “good manufacturing practices”, and those types of manufacturing processes provide still more definition and clarity to the term “sized”, as interpreted and applied in the world of medical devices.

The disclosures herein focus on implants for tibial and meniscal cartilage replacement, and do not specifically address implants that are sized to replace the rounded “runners” at the bottom end of a femur (i.e., a thigh bone). In many cases where tibial or meniscal cartilage has been damaged by injury or disease, the damage that has been suffered by the tibial and/or meniscal surfaces will cause or aggravate abrasion of either or both of the femoral runners, and arthroscopic replacement of either or both of the femoral runners will also be required. However, femoral implants designed for arthroscopic insertion are described in other sources, including PCT patent applications WO 03/103543, WO 03/103543, and WO 05/032426 (all by the same inventor herein). and U.S. Pat. No. 6,994,730 (Posner 2006). Since rounded implants for replacing femoral runners are disclosed in various other sources, that information is not repeated herein.

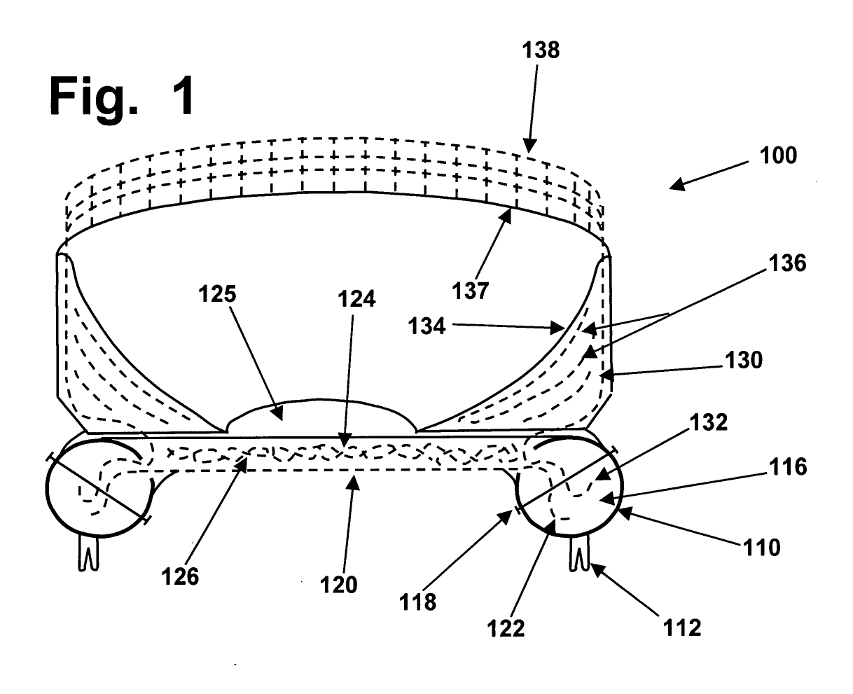

Accordingly, referring to the drawings, FIG. 1 discloses a knee repair implant 100, comprising several main components that can be regarded as subassemblies, listed and described below.

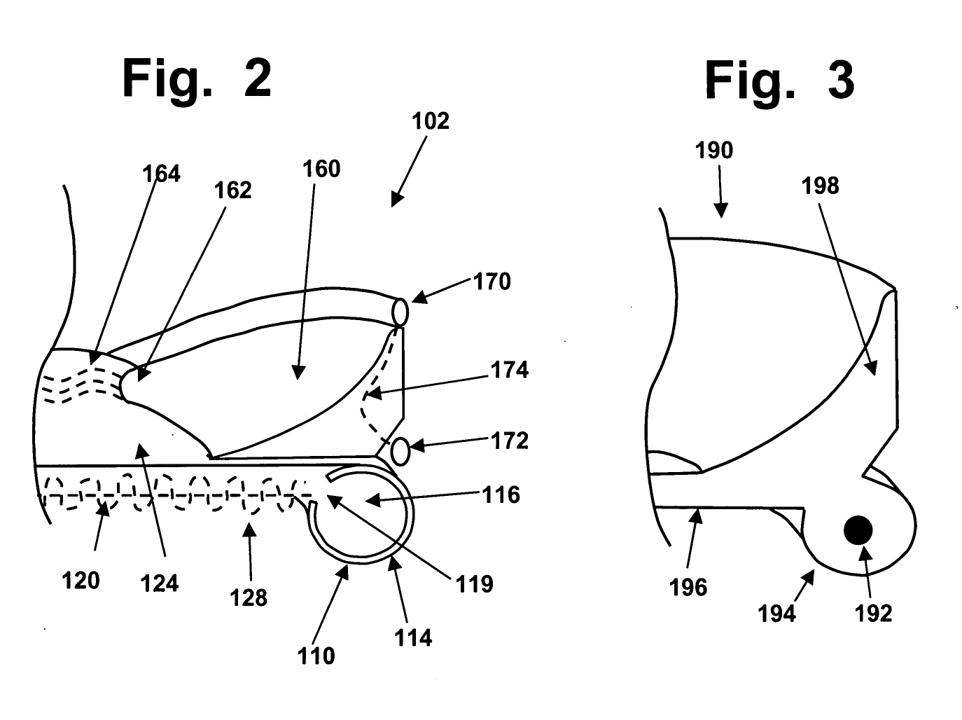

The dashed lines in FIGS. 1 and 2 are intended to represent porous fabrics or comparable materials, which can be woven, knitted, embroidered on a backing layer which is then dissolved and removed, or created by any other suitable means (such materials also can be made of, for example, layers of synthetic polymers that are designed to be easily penetrated by a needle, staple, or other device for securing purposes). For convenience, any such materials that would normally be regarded as “single layer” materials are referred to herein as “fabrics”. If such materials are created or modified in ways that provide additional layers and/or thickness (such as tufted materials, materials made by three-dimensional weaving, etc.), the resulting thicker materials can be referred to by terms such as fiber arrays, meshes, matrixes or matrices, etc. In general, it should be recognized that most types of fabrics or other fiber arrays are designed and made in ways that allow them to be easily penetrated by sutures, staples, pins, rings, rivets, or other devices, for anchoring or other securing purposes.

The design and structure of the implants illustrated in FIGS. 1-1 can be explained most easily and clearly by starting at the bottom, and moving upward. As used herein, any directional terms (such as bottom and top, lower and upper, etc.) assume that a patient who is receiving a knee implant will be lying on his or her back, with the leg bent at a right angle at the knee, so that his or her tibial (shin) bone is vertical, with the tibial plateau cartilage in a horizontal orientation at the top of the tibial bone. The surgeon will be working on the knee using arthroscopic instruments, including a light source and a miniature video lens, with a steady flow of clear saline solution being pumped through the operating field to keep things visible.

The bottom component of implant device 100 as shown in FIG. 1 is an anchoring rim 110, with a plurality of anchoring pegs 112 affixed to rim 110 at spaced locations around the rim.

As used herein, when referring to an implant, the term “rim” refers to an outer edge which is provided with at least one relatively hard and/or stiff component, such as a component made of a nitinol allow or other shape-memory material. This would exclude, for example, the edge of a piece of fabric or similar flexible material that is affixed to a bone or other tissue by a series of pins, sutures, staples, or similar fixation means; however, it would include a rim assembly that is covered by a flexible polymer and internally reinforced by a ring of shape-memory material, as illustrated in FIG. 3.

A rim, as that term is used herein, does not need to form a completely closed ring, polygon, or similar structure. However, in most cases of implants for repairing cartilage in joints, the rim preferably should be largely or even fully continuous and enclosed, to provide maximum strength, stability, and durability. In a field of use such as described herein, as a rim device is lengthened and extended in ways that enclose progressively greater portions of the area of an implant base, it generally can provide higher levels of strength, stability, and durability, culminating in a fully-enclosed rim providing the highest level of strength, stability, and durability that can be provided by any rim which is limited to fixed and constrained cross-sectional and overall dimensions. Accordingly, a rim segment or component that merely provides, for example, a single straight segment along one side of a rectangle or other polygon, generally should be avoided. However, to avoid creating a situation where imitators and competitors would have strong incentives to avoid the patent claims by making and selling inferior products, or by using artifice and pretense (such as, for example, by dividing the rims of competing implant devices into segments that would remain separate during manufacture and shipping, and that would be connected only during assembly by a surgeon), the term “rim” as used herein is intended to cover any type of continuous hard and/or stiff component affixed to one or more outer edges of an implant device, for the purpose of provided more secure anchoring of the component to bone or other tissue.

In interpreting and applying that definition, the phrase “outer edge” also needs attention. For the strongest, safest, and most secure anchoring, a working presumption arises that an anchoring rim preferably should fully surround and enclose the entire “base” of an implant (which is defined herein to refer to those components of an implant that will press against a bone surface). This presumption arises from the concern that other designs which might place one or more parts of the base outside of an anchoring rim would create a risk that one or more such “outside the rim” parts might be pried up and worked loose, either by repetitive motion of the joint, or by an accident that otherwise might not cause failure of a well-designed joint (this relates back to a similar concern that arises over the “central anchoring” approach exemplified by the Posner '730 patent, as described in the Background section).

However, despite that working presumption, a concern arises that if “rim” is defined herein in a way that is narrowly limited to only the outermost periphery, it would create a situation where imitators and competitors would have an incentive to avoid the patent claims by placing some relatively small portion or component of the base outside the rim.

To minimize that temptation and avoid that risk, the term “outer edge”, as applied herein to an anchoring rim, is given an arbitrary definition that is pegged to a 75% number, applied linearly. An anchoring rim or other boundary-type component is regarded herein as being positioned around or along the “outer edge” of the base of an implant device, if the outermost surface of the rim or similar component is positioned, on average, at least 75% (i.e., at least ¾) of the distance toward the outermost periphery of the base, when measured linearly from the centerpoint of the base. If the base has an irregular shape, the centerpoint will be the “centroid” of the base; in most cases, this would be the center of gravity of the base when the rim is not attached. It should be emphasized that that is an arbitrary definition, and in most cases, good design principles will indicate that the base should be on the outer periphery, rather than merely adjacent to it, to the fullest extent possible.

In accord with those design principles, anchoring rim 110 as shown in FIGS. 1 and 2 is positioned around the outer periphery of the implant base 111. It comprises an outer ring 114, which largely encloses and securely grips a flexible component 116 made of a synthetic polymer (referred to herein as polymer insert 116).

If desired, the outer ring 114 of anchoring rim 110 can be made of a shape-memory material that will shrink substantially when chilled to the temperature of “saline slush” (i.e., physiological saline solution that has been chilled to a borderline freezing temperature). Such shape-memory materials (including a class of metal alloys that are often referred to generically as “nitinol” alloys) are known, and are described in books such as Otsuka and Wayman, editors, Shape Memory Materials (Cambridge Univ Press, 1999), and at websites such as www.smst.org and www.nitinol.info, which are run by an organization called Shape Memory and Superelastic Technologies (SMST). The use of shape-memory materials in medicine and surgery is described in D. Stoeckel, “Nitinol Medical Devices and Implants”, presented at the SMST 2000 Conference and available from websites such as www.nitinol.info/pdf_files/stoeckel_{—}1.pdf. The use of such alloys in anchoring rims that contain flexible polymer inserts is described and illustrated in more detail in PCT patent application PCT/US05/43444, by the same Applicant herein; the contents of that PCT application are incorporated by reference herein.

It should be noted that not all elastic materials qualify as “shape-memory materials”. For example, a rubber band is elastic, but it can be laid on a flat surface in any of various shapes, in relaxed form and with no internal stresses, so long as it is not being stretched. By contrast, a device made of a shape-memory material will seek to return to a specific manufactured shape, whenever any external forces and stresses are removed from it, so long as it is not being subjected to temperatures that are above or below a “transition zone” that will cause it to change shape.

The use of a shape-memory material that will shrink when chilled, to form the outer ring 114 of anchoring rim 100, can enable a surgeon to manipulate and shrink an implant device, during surgical insertion of the device 100 into a knee (which normally will involve pushing the device into the joint through an arthroscopic insertion tube). This can minimize the amount of cutting and tissue displacement that is required, thereby minimizing the damage to tissues and blood vessels that sit in or near the insertion pathway. After the implant device has been inserted into a joint, it will warm up to body temperature, causing it to expand and return to its manufactured size and shape. That size and shape will enable the anchoring rim 110, made of shape-memory alloy or similar material, to fit into a groove that has been created in the tibial bone surface by using specialized tools and guides, as described below.

However, it also should be noted that shape-memory and/or superelastic materials do not always require or use chilling and rewarming steps that cross a transition temperature. Instead, devices such as metal alloy rings made of these types of materials can be highly useful in implants that: (i) will be rolled, squeezed, or otherwise manipulated in ways that will allow them to be pushed through arthroscopic insertion tubes; and then, (ii) will return to their manufactured shape after insertion has been completed, so that they can be properly installed inside a joint that is being repaired.

If a shape-memory rim is used to grip and mostly enclose a polymer insert 116, as shown in FIGS. 1 and 2, the polymer insert 116 can be molded in place, inside the outer ring 114, by convention means, such as by injecting a pre-polymer liquid that will cure (or set, harden, or similar terms) into a solid but flexible, resilient, rubber-like polymer. If desired, polymer insert 116 can be made of the same type of polymer that provides the cartilage-replacing components discussed below, or it can be made of a different polymer type.

In the embodiment illustrated in FIG. 1, polymer insert 116 has been molded around the ends 122 and 132 of reinforcing fabrics 120 and 130, which are made of very strong fibers or other suitable thin-layer porous polymeric material. As mentioned above, reinforcing fabric layers 120 and 130 can have woven, knitted, embroidered, tufted, stitched, or comparable structures known to those skilled in the art. If desired, a plurality of pins or staples 118 can be inserted through the secured ends of reinforcing layers 120 and 130, using holes provided at spaced locations around outer ring 114, to further strengthen the attachment of reinforcing layers 120 and 130 to anchoring rim 110. Regardless of whether such pins or staples are used, molding a strong polymer insert 116 around the edges of reinforcing layers 120 and 130, inside a ring 114 made of a strong alloy or similar material, will securely affix the fiber layers 120 and 130 to the anchoring rim 110, provided that the slot, gap, or other opening 119 in ring 114 is sufficiently narrow and constricted (such as less than about 45 degrees, and preferably less than about 30 degrees in arc).

Reinforcing layer 120 should be designed to maximize and optimize its ability to reinforce a hydrogel or other polymer layer 124. Accordingly, any of several options can be used to create substantial thickness for the reinforcing layer, in the portion of the reinforcing layer that is surrounded by anchoring rim 110. As an example, a computer-controlled stitching machine can be used to create a tufted or stitched layer 126, which will create substantial thickness (which can also be referred to as depth, height, volume, etc.) for reinforcing layer 120. Computer-controlled stitching machines that can create tufted or stitched layers in pre-programmed patterns, with controllable heights and densities, are known, such as the AMAYA(TM) embroidery machine, made by a company called Melco (www.melcousa.com), a subsidiary of the Saurer company (www.saurer.com). If stitching or tufting is used, the fabric layer that holds and supports the tufts or stitches is usually called a “backing” layer.

Polymer layer 124 will replace the thin layer of hyaline cartilage that normally covers the surface of a tibial plateau. This polymer layer 124 can be a hydrogel (i.e., a polymer that will absorb and hold water, when saturated with an aqueous liquid such as a physiological saline solution) or a non-hydrogel (or any combination thereof, such as a layered structure), provided that the selected polymer(s) must have a suitable combination of: (i) physical traits that emulate the non-rigid flexibility and resilience of healthy natural cartilage; and, (ii) sufficient strength and durability to last for decades, when used in a cartilage-replacing implant in a joint such as a knee. Two classes of high-strength non-rigid polymers that can meet those requirements (in either hydrogel or non-hydrogel form) can be made from polyurethanes, and polyacrylonitriles; other types of polymers also can be evaluated for such use, if desired.

The chemical terms “polyurethane” and “polyacrylonitrile” refer to certain types of chemical linkages that are formed in the “backbone” chains of polymers, when certain types of reagents )often called monomers) are chemically converted into polymers. There are numerous ways to modify the other constituents of such polymers, such as by controlling the spacing, lengths, and end groups of various “side chains” or “pendant moieties” that will form “bridges” that will attach the polymeric backbone chains to each other, in ways that form flexible and resilient three-dimensional networks of molecular chains in a flexible polymer. These and other approaches can be used to create various types of strong, non-rigid, hydrophilic formulations with either urethane or acrylonitrile linkages in the polymerized chains, and such polymers can be made in either hydrogel or non-hydrogel form. These aspects of polymer chemistry (and methods for molding and curing polymer-forming reagents into flexible devices that can be reinforced by fabrics or other fiber arrays having very high strength) are known to those skilled in the art, and are discussed in detail in numerous books, articles, and patents.

The upper surface 125 of polymer layer 124 must provide a smooth and wettable articulating surface. This implies that reinforcing layer 120 (including any tufted, stitched, or other structures that may be part of that layer) should not reach, penetrate, or disrupt the upper surface 125, which should be covered by a completely smooth layer of polymer.

In addition, upper surface 125 preferably should have a negative electrical charge that approximates the charge density of healthy natural cartilage. This type of negative charge can promote desirable interactions between polymer surface 125, and certain components of synovial fluid, which lubricates the joint. This type of negative charge on a polymer surface can be created by chemical treatment (such as mild sulfonation or fluorination) of a polymer surface, or by incorporating sulfur, fluorine, or other electronegative compounds into a polymer as it is being created.

The bottom of polymer layer 124 preferably should have a porous structure that will promote ingrowth of bony or scar tissue into the anchoring surface of implant 100, during a span of weeks or months following surgery. This type of tissue ingrowth will create stronger and more durable anchoring of the implant 100 to the tibial bone. The use of tissue ingrowth for stronger anchoring of surgical implants is well-known, and many types of orthopedic implants are designed to promote it.

FIG. 1 is a simplified view, which indicates that reinforcing layer 120 can provide the tissue ingrowth layer on the bottom of polymer layer 124. FIG. 2 is a more detailed view, which illustrates a tufted or stitched layer 128 that extends below fiber layer 120 (which thereby causes the main fiber layer 120 to serve as a “backing layer”), in a manner that provides loop tips that extend, in an exposed manner, below the bottom surface of the polymer layer 124. These exposed loop tips (which can be present in densities of hundreds of loops per square inch) form a porous surface, and they can be made of biocompatible fibers that will actively promote tissue ingrowth into the bottom surface of an implant device. Alternately, various other types of porous and flexible biocompatible materials can be created (such as screen-type meshes made of very thin strands of certain metals or alloys) which can promote tissue ingrowth into the bottom (anchoring) surface of an implant.

FIG. 1 indicates that meniscal segment 134 (which has a wedge shape when viewed in cross-section, and an arc shape when viewed from above) is secured to anchoring rim 110 by means of fibrous reinforcing layer 130, a portion of which is embedded within polymer segment 134.

Meniscal segment 134 preferably should be additionally reinforced by one or more layers of fibrous material having substantial thickness, which can be referred to by terms such as mesh, matrix, fiber array, etc., as indicated by dashed lines 136 inside meniscal segment 134. This type of three-dimensional reinforcing material can be made by various known methods, such as three-dimensional weaving, which is used by companies such as TechniWeave (a division of Albany International) to make high-strength composite materials.

At least one layer of reinforcing fabric 138 should extend upwardly, above the upper rim 137 of meniscal segment 134. This reinforcing segment 138 (which may be, but which is not required to be, an extension of the same reinforcing layer 130 which is secured inside of anchoring rim 110) will provide an upper anchoring component for implant 100. It is designed to be sutured, stapled, or otherwise affixed to either or both of two types of soft tissue: (i) the tendons and other tissues that form the capsule, which surrounds a knee joint and holds in the synovial fluid; and/or, (ii) any remnant (or residual) tissue that has been left in place, from the outer rim (or periphery) of a meniscal segment that was partially resected and removed.

Accordingly, fabric segment 138 provides an upper anchoring means for securing the implant, around its upper perimeter, to soft tissue. This upper anchoring system, affixed to soft tissue, will work in coordination with anchoring rim 110, which is anchored to hard bone. By working together, the upper and lower systems can provide better anchoring for meniscal segments than any prior system previously disclosed in any prior art, due largely to two important contributing factors. First, this anchoring system closely emulates the normal and natural anchoring systems of healthy meniscal segments, in knees. And second, the anchoring systems used herein will act around the enlarged periphery of an implant, rather than in its center. As a result, these two different anchoring systems can provide a larger, stronger, more durable interface, which will be much less vulnerable to the types of “bending moment” problems described in the Background section, which used the analogy of an enlarged steering wheel to provide a driver with a better and stronger grip than can be obtaining by trying to grab and hold a relatively narrow shaft.

The height of the polymeric meniscal segment 134, as illustrated in FIG. 1, is exaggerated, partly to make the cross-sectional depiction less crowded, and partly to emphasize the role of a meniscal segment in created a rounded bowl-shaped surface that will accommodate and support the rounded surface of a femoral runner. In reality, the meniscal segment is substantially thicker in its middle section, and it tapers off toward both ends of the arc-shaped segment.

As mentioned above, a plurality of anchoring pegs 112 can be affixed to anchoring rim 110, at spaced locations around the rim. These pegs will be designed to be pressed into accommodating anchoring sleeves (which also can be called barrels, cylinders, or similar terms). The anchoring sleeves preferably should be emplaced in holes that have been drilled into hard bone, with the aid of one or more types of guides or templates, near the start of an operation, before the cartilage-replacing implant device is inserted into the joint. A sawtooth-like surface on the outside of an anchoring peg will engage an accommodating sawtooth-like surface inside an anchoring sleeve, in a manner that effectively locks the peg in place, in an anchoring sleeve, once it is pressed firmly into place. This approach to installing an implant will provide a surgeon with better access and more room to work, before the available space in an exposed knee joint is occupied by an implant device. This approach is described in more detail in PCT application WO 05/032426 (by the same Applicant herein), the contents of which are incorporated herein, by reference, as though fully set forth herein.

If desired, alternate anchoring means can be used to secure an anchoring rim to a bone, at a plurality of locations around the length of the rim. For example, a set of anchoring pins or screws can be driven through an accommodating set of holes, eyelets, or similar orifices or appurtenances at spaced locations around the rim. However, it should be recognized that enlarged anchoring pegs having sawtoothed cylindrical outer surfaces, which will fit into accommodating sawtoothed surfaces inside the barrels of anchoring receptacles, can provide greater strength, stability, and durability, than pins or similar devices. This greater strength and durability arises from both: (i) the larger interface areas that can be provided by enlarged anchoring components, compared to narrow-diameter anchoring pins; and, (ii) the ability of locking sawtooth or other irregular surfaces to provide better gripping and “purchase” than the relatively smooth surfaces of anchoring pins, which are comparable to small nails.

FIG. 2 is a cross-sectional cutaway depiction of one edge of a modified implant device 102, having a meniscal segment 160 which is shown in a cutaway view near one of its two arcuate tips 162. This meniscal segment 160 has several anchoring components not shown in FIG. 1. First, a strip of fabric 164 or similar flexible material, which emerges from arcuate tip 162, enables the arcuate tip 162 to be anchored to a selected tissue location, such as a tibial spine (presumably via an anchoring pin or similar device), a ligament that emerges from a tibial spine, a portion of a knee capsule, or a meniscal rim that was partially resected in a manner that left behind a suitable remnant. In addition, upper and lower anchoring rings 170 and 172 (which can also be called hoops, rims, etc.) are provided, above and below the outer periphery of meniscal segment 160. These anchoring rings 170 and 172 are made of a nitinol-type alloy or similar shape-memory material, and they are sized and shaped to press, in a relatively gentle and non-abrasive manner, against the inner surface of the capsule that surrounds and encloses a knee joint. Upper ring 172 can be anchored (by sutures, staples, etc.) to the inner surface of the capsule, in addition to (or instead of) an upper segment of porous fabric 138 as shown in FIG. 1. Lower ring 174 also can be anchored to the inner surface of the capsule, if desired; however, its proximity to anchoring rim 110 may render such a lower attachment unnecessary, and might interfere with proper sliding motion of meniscal segment 160 on top of tibial plateau segment 124. A porous reinforcing device 174, made of a woven or knitted fabric, a metallic screen, a perforated plastic layer, or other comparable material, also can be embedded within meniscal segment 160, in a manner that spans the distance between upper ring 170 and lower ring 172, to provide additional reinforcement for the rings and for meniscal segment 160.

FIGS. 1 and 2 illustrate polymeric meniscal segments 134 or 160 that are separate from the polymeric layers 124 that will replace hyaline cartilage on a tibial plateau. This enables a meniscal segment to move and slide (in a limited and constrained manner) across a thin film of synovial fluid that will lubricate the interface between the lower surface of the meniscal segment 134 or 160, and the upper surface of polymer layer 124.

After implantation, any sliding motion by meniscal segment 134 (or 178), relative to polymer layer 124, will be limited and constrained by at least two and possibly three sets of attachments. In all cases, the entire lower edge of reinforcing fabric 130, shown in FIG. 1, will be securely and permanently affixed to anchoring rim 110. In addition, the upper anchoring segment 138 (or the upper anchoring ring 172, in FIG. 2) will be affixed to either or both of two types of soft tissues: (i) the tendons and other tissues that form the capsule which surrounds the knee; and/or, (ii) any remnants that are left in place from the periphery (or rim, margin, etc.) of a meniscal segment that was partially but not completely removed. It should be noted that those two anchoring modes are on spaced and opposed sides of a meniscal segment. This approach to positioning two anchoring components, above and below the elongated wedge-shaped flexible polymer segment, can be referred to by terms such as flanking, bracketing, etc. Those types of flanking or bracketing anchors, along the upper and lower edges of the outer periphery of an elongated flexible wedge, is believed to provide very good potential for anchoring a synthetic meniscal replacement in a knee joint, and is believed to provide an improvement over all meniscal anchoring systems in the prior art that are known to the applicant.

In addition to upper and lower anchoring modes mentioned above, FIG. 2 shows a fabric or similar flexible material 164, emerging from tip 162 of meniscal segment 160, as described above.

Accordingly, the modes of attachments disclosed herein are believed to provide higher and better levels of stability and security than any prior known meniscal implants, while also emulating in many respects the normal anchoring of native meniscal segments.

FIG. 3 illustrates an implant device 190 which has certain alternate design features that merit attention, First, this design comprises an anchoring ring 192 made of a nitinol-type alloy or other shape-memory material, embedded within a flexible and resilient polymeric anchoring rim 194. The shape-memory ring 192 will cause the anchoring rim 194 to return to a stable and predictable size and shape (which can fit into a groove that has been prepared in a bone surface), after any bending stresses that were imposed on the device, during insertion, have been relaxed.

If implant 190 is intended to replace labral tissue in a hip or shoulder, polymer layer 196 normally would be designed to replace a concave layer of hyaline cartilage, in a ball-and-socket joint. Accordingly, in such an implant, polymer layer 196 likely would be provided with a concave upper surface, to emulate the socket-shaped natural cartilage layer that will be replaced.

It also should be noted that there is no distinct boundary layer or slidable interface between the relatively flat polymer layer 196, and the wedge-shaped component 198 that generally rises above layer 194. As mentioned elsewhere, because these types of prosthetic replacements preferably should emulate natural physiological structures, a presumption arises that this design (with no slidable interface between wedge 198 and layer 196) likely will be preferred for labral replacements in hips and shoulders, but not for meniscal replacements in knees. However, that is merely a working presumption, and testing of both types of structures, in all three types of joints (knees, hips, and shoulders) should be carried out at appropriate times.

Along those lines, it should be kept in mind that implants designed for different classes of patients may have different optimal designs. For example, a knee implant for an elderly patient who does not and will not run or jog, for exercise, may have a different preferred design than an implant for a young patient whose knee was injured in an automobile or sporting accident.

In addition, even if a predominantly slidable interface is preferred for meniscal replacements for knee joints, such a slidable interface can be limited to only a portion of the length of an arc-shaped meniscal replacement, and a molded or otherwise bonded interface that does not allow any sliding motion between layer component 196 and arc-shaped component 198 can be provided at either or both of the tips of the arc-shaped component 198. This type of molded or bonded interface can provide a potentially useful mode of indirectly but securely anchoring the tips of an arc-shaped component to a larger implant, and to its anchoring rim.

### Arthroscopic Tools for Preparing Bone Surfaces

Attention must now be turned to a set of arthroscopic tools that will enable a surgeon to properly prepare a tibial bone surface, so that the bone will be ready to receive a knee-repair implant device as described above.

Accordingly, the next section describes a new type of planing tool that is designed to create a generally flat surface, of the type that will be required for an implant that is affixed to a tibial plateau.

After that, the following section (with its own subheading) describes an expanded class of planing tools that can be used to prepare rounded or otherwise curved bone surfaces, such as appear on the lower surfaces of a femoral runner.

Then, the next section (with its own subheading) describes a new type of tool designed to create a groove in a bone surface, in a manner that provides the groove with both (i) a consistent and controllable depth and width, and (ii) a predetermined overall size and shape, both of will enable the groove to receive and accommodate the anchoring rim of an implant, such as anchoring rims 110 or 192 as shown in FIGS. 1-3.

After those types of tools have been used, the prepared bone surface will be ready to receive a cartilage-replacing implant as described above. The groove will help stabilize and reinforce the anchoring rim of the implant, and the planar or curved bony tissue surface can begin growing into the porous bottom surface of the implant. Both of those interactions will lead to stronger, more stable, and more durable anchoring of these implants.

It should be noted that any types of suitable planing and/or groove-forming tools which are previously known, disclosed herein, or hereafter discovered, can be used to prepare a bone surface to receive an implant as disclosed herein. Accordingly, these implants are not limited to use on bone surfaces that have been prepared by any particular type(s) of tools.

Nevertheless, the tools described below can be highly useful for such bone preparation work, and therefore, their disclosure herein is deemed necessary, to fully satisfy the requirement for disclosing the best mode of carrying out the invention.

Beyond that legal requirement, it is hoped that interested readers will realize that the complete set of inventions and disclosures by this applicant, herein and in various other patent applications, have crossed a threshold and reached a “critical mass”. A number of lines of interrelated technology that will enable major advances in surgical repair of cartilage have moved into place, and are ready for a balanced, coordinated, multi-part development and testing program.

### Planing Tools for Creating Smooth Surfaces

Patents on tools for preparing bone surfaces for receiving implants can be found in several subclasses (such as 79, 80, and 96) of Class 606; examples include U.S. Pat. No. 5,817,095 (Smith 1998), U.S. Pat. No. 6,120,507 (Allard et al 2000), U.S. Pat. No. 6,358,253 (Torrie et al 2002), and U.S. Pat. No. 6,884,246 (Sonnabend et al 2005). However, most such art involves items that are either: (i) designed to drill or ream holes, or other non-planar sculpting of bones surfaces; or, (ii) designed for use in “open joint” rather than arthroscopic surgery. The Applicant herein is not aware of any patents or articles describing tools having designs comparable to those shown herein, to facilitate the arthroscopic preparation of a smooth (or “planed”) bone surface.

As used herein, terms such as “planed” or “planar” refer to a bone surface that-has been treated (or prepared, worked, machined, or similar terms) in a manner that provides it with a relatively smooth and regular surface (i.e., substantially smoother than the surface that existed prior to the treatment). The reference to “smooth and regular” indicates that a prepared bone surface should be free of burrs, crevices, protrusions, or other irregularities that, if present, would likely interfere with secure and stable anchoring of a pre-manufactured implant device to the bone surface.

The term “planing tool” is used herein to refer to a tool with at least one rotating component having a cutting, grinding, abrasive, or similar surface, designed for creating relatively smooth and regular surfaces on bones that are being surgically prepared to receive an implant device. The requirement for “at least one rotating component” excludes, for example, devices such as knives, scalpels, and other cutting blades that are not part of a machine-type tools, and spatulas or similar devices that might be used to press against or otherwise smooth out a filler-type material (comparable to a dental filling material used for repairing teeth) that is being used to fill and occupy or otherwise treat a void, crack, or other defect in a bone surface.

As used herein, terms such as “planar” or “a planed surface” are not limited to the mathematical definition of a plane, which refers to a flat and non-curving surface. Instead, planing tools can also be used to prepare smooth rounded bone surfaces (as occur on femoral runners, in ball-and-socket joints such as hips or shoulders, etc.), so long as the curvature and shape of the prepared bone surface can be controlled in a manner that will match and accommodate a known curvature of an anchoring surface of an implant device.

To simplify the discussion below, any directional references used herein (such as upper, lower, top, bottom, above, below, etc.) assume that a planing tool will travel across the upper surface of a horizontal bone surface, with the exposed grinding surface of the burr positioned on the bottom (lower) side of the tool, and with the cowl positioned above the burr on the top (upper) side of the tool. In practice, when a patient is having his or her knee worked on, he or she normally will be supine (lying on his/her back), with the knee flexed (i.e., bent), so that the tibial plateau will be roughly horizontal, as described herein.

It should also be noted that during a typical arthroscopic operation, clear saline solution is continuously pumped into the joint, through a tube (often called a cannula) via an outlet that usually is positioned adjacent to a light source and a miniaturized video lens. The saline liquid passes through the operating field, and is removed by a suction tube, along with any entrained blood, bone chips, cartilage flakes, and other particles or debris. This allows the surgeon to see the operating field more clearly, on a video monitor. Accordingly, the tools described herein preferably should be equipped to provide a suction conduit that will receive and remove any such liquid, blood, and particles from the joint.

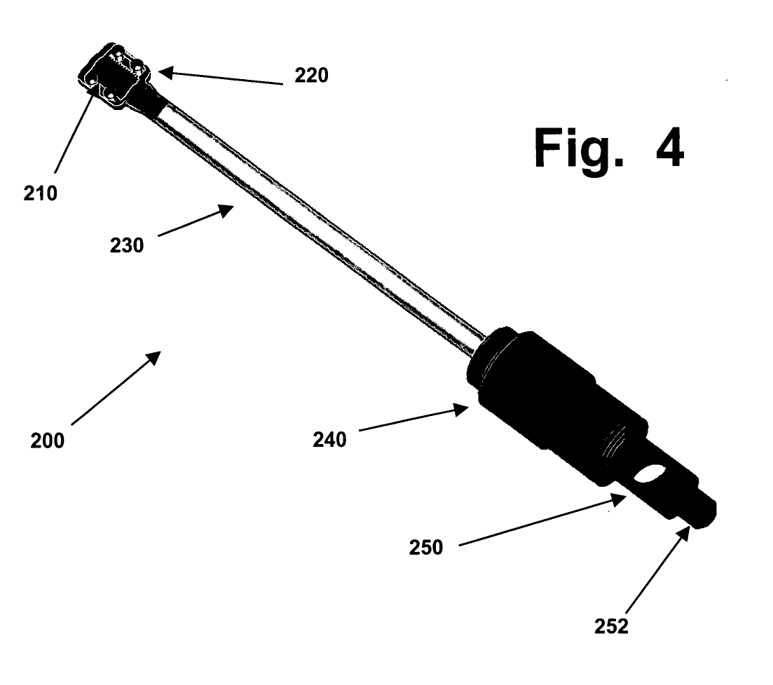

Accordingly, one embodiment of an arthroscopic tool 200 designed for use in planing a tibial plateau surface is illustrated in FIGS. 4-6. The main components of tool 200 include a grinding burr 210, a protective cowl 220 with adjustable feet 222, a non-rotating sheath 230 which encloses a rotating driveshaft 232, a handle component 240, and a drive interface 250. Each of those components is described in more detail below.

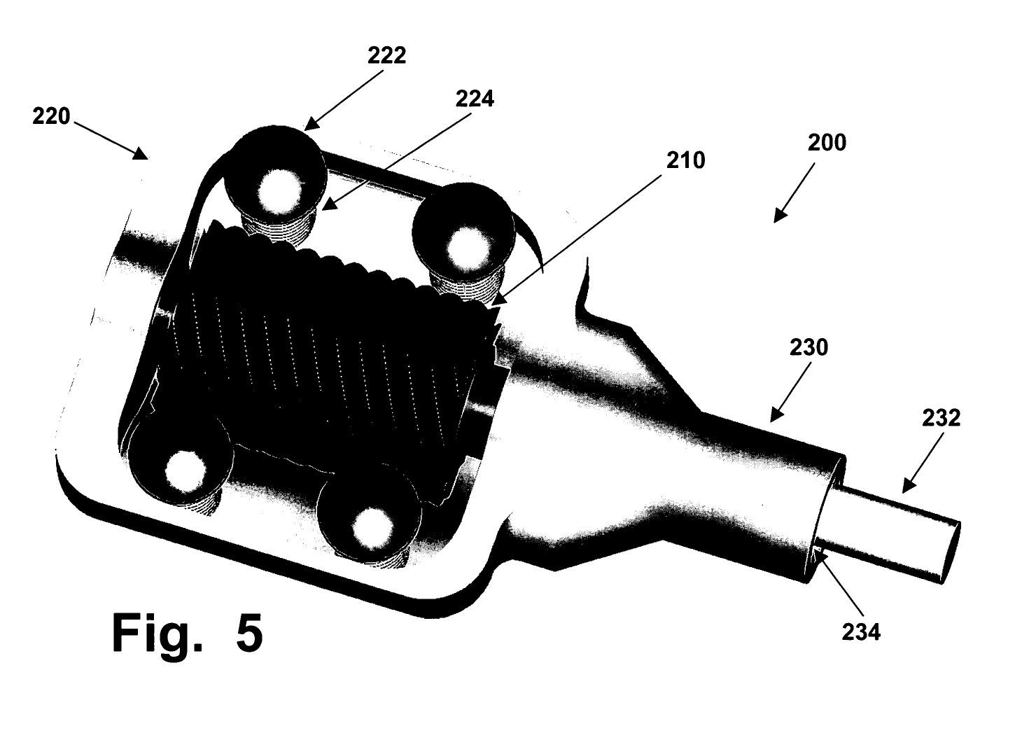

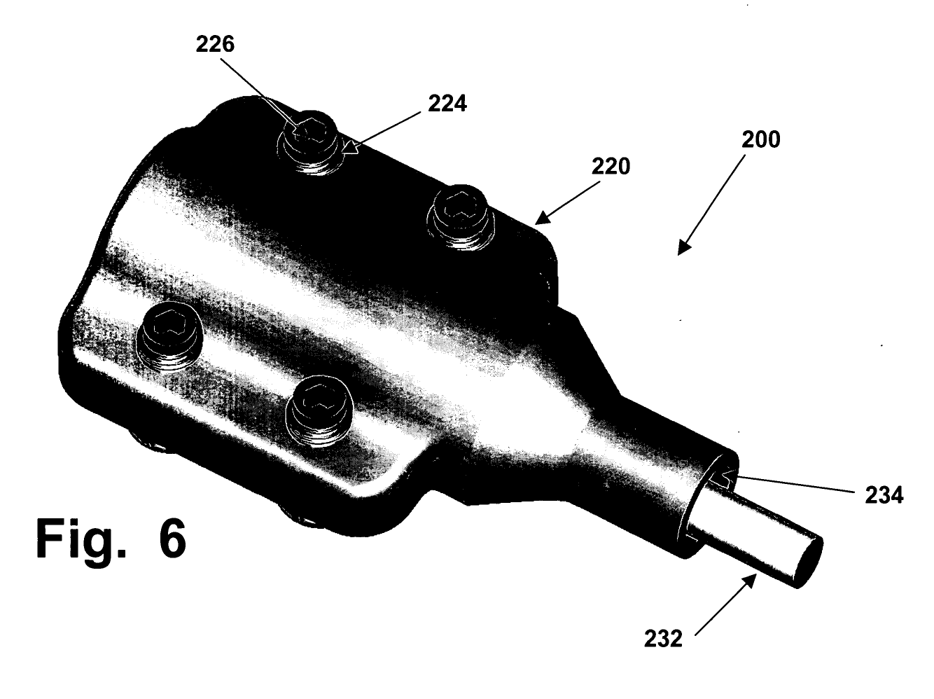

The “working end” of planing tool 200 is illustrated in more detail in FIG. 5 (which shows the underside) and FIG. 5 (which shows the top side of the cowl). As shown therein, burr 210 has a cylindrical working surface, formed of numerous tiny but sharp angled ridges. A preferred arrangement of such ridges uses a helical orientation, as illustrated, for two reasons: (i) the angled orientation of the helical ridges can minimize the risk and frequency of disruptive “catching” of the burr, while it is being used to grind and smooth a hard bone surface; and, (ii) when the helical ridges spin, they will generate pumping-type forces that will help drive saline solution, blood, and particles toward the inlet of a suction tube, represented by annular gap 234 in FIGS.5 and 6.

An assortment of planing tools with helical ridges as shown, but with varying heights and roughness (comparable to having an assortment of sandpapers with coarse, medium, or fine grit) preferably should be made available to surgeons, and other types of cutting, grinding, or abrasive surfaces also preferably should be made available, for use during different stages of an operation.

The speed of rotation of the burr also should be controllable, using a device such as a foot pedal that can be operated by the surgeon. Drive machines which will provide that type of power and control are readily available from various suppliers, and handle 240 and drive interface 250 (which includes a non-round component 252 that fits into an accommodating slot in a connector device at the end of a flexible drive cable) preferably should be directly compatible with drive units that are already in use in hospitals where most orthopedic surgeons work. Such drive units provide flexible cables with “quick connect” devices at their ends, containing flexible driveshafts which rotate inside the non-rotating sheaths that surround and enclose the drive cables.

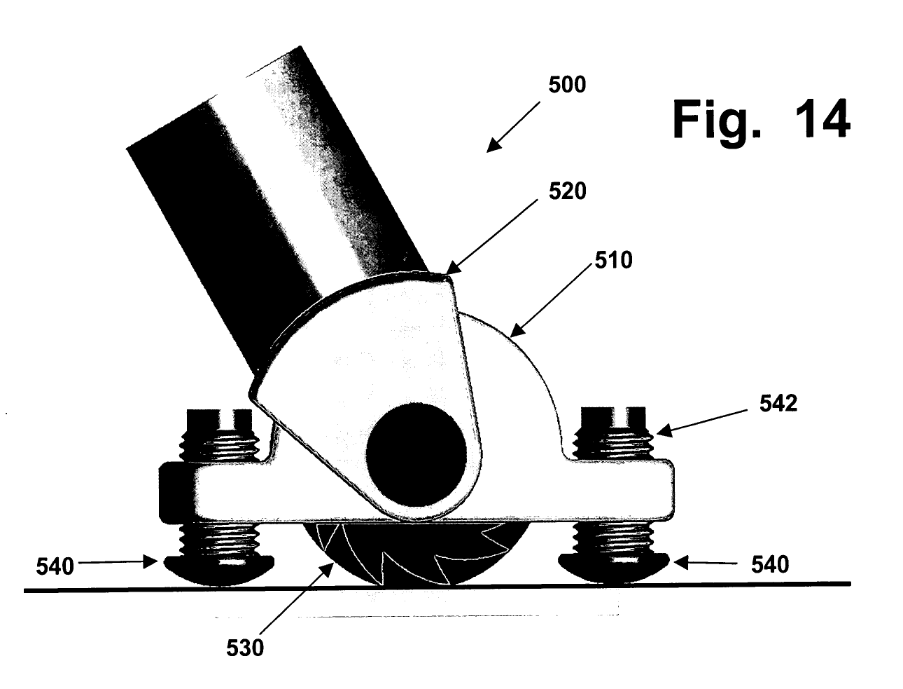

FIGS. 5 and 6 also illustrate a set of four smooth-surfaced “feet” 222, which are mounted on the ends of threaded (or otherwise adjustable) shafts 224. Using means such as hex sockets 226 at the ends of threaded shafts 224, the height (or extension, retraction, etc.) of any or all of the feet 222 can be adjusted, relative to cowl 220 and the surface of burr 210. As can be envisioned by looking at the cutaway side view in FIG. 14, if the feet are extended to a point slightly below the lowest tangential edge of the grinding burr, the tool will make a convex cylindrical surface. Conversely, if the feet are retracted to a point slightly above the lowest tangential edge of the grinding burr, the tool will make a concave cylindrical surface. At the current time, it is presumed that in a tibial plateau, the prepared bone surface should be flat. However, slightly concave or convex bone surfaces may help stabilize tibial implants and increase their durability, and those approaches should be modeled and tested at an appropriate time.

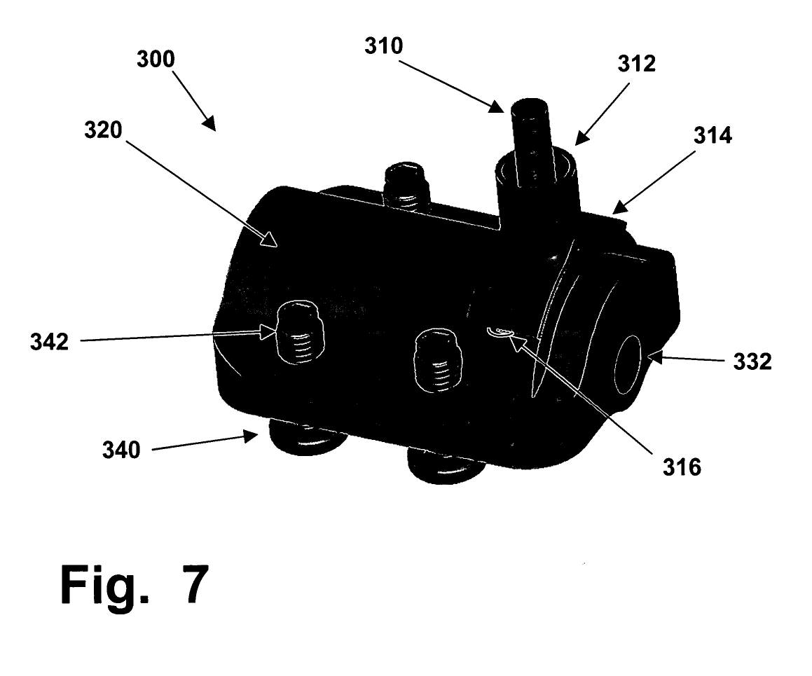

Curvature is crucial in femoral runners. Accordingly, a different class of planing tools, designed mainly for preparing femoral runner surfaces but which also can be used for other uses whenever appropriate, is illustrated in FIGS. 7-14. In one embodiment, shown in FIG. 7, planing tool 300 has a driveshaft 310 inside a non-rotating sheath 312, mounted perpendicular to axle 332 of grinding burr 330. The driveshaft sheath 312 is affixed to a curved plate 314, which will slide across the generally cylindrical upper surface of cowl 320, traveling in a curvilinear slot 316, which is illustrated in more detail in FIG. 10. This will provide a surgeon with control over the travel and orientation of tool 300, since the “handle” end of sheath 312 will be accessible at all times, outside the joint being repaired. If desired, the “handle” end of access tube 312 can be coupled to any of various types of gripping and rotating devices or handles, or to computer-controlled devices containing “servo” or similar mechanisms that can carry out a pre-programmed bone preparation.

FIG. 7 also depicts feet 340, mounted at the ends of threaded or otherwise adjustable shafts 342 having hex sockets. As discussed above, when feet 340 are extended or retracted, they will control the curvature of the bone surface being prepared, as can be visualized by considering the side view in FIG. 14.

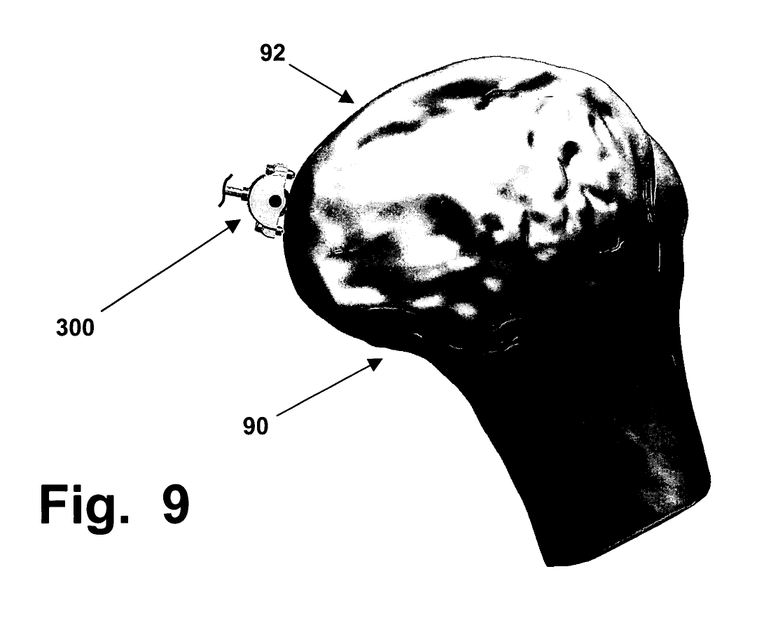

In actual use, it would be preferable to provide an electromechanical system that will allow a surgeon to adjust the extension of the shafts 342 from outside a joint, while the operation continues without interruption. An externally-controlled feet-adjusting mechanism can help a surgeon more easily create the proper curvature on a femoral runner, which has a “cam” type curvature rather than an exact round curvature, as illustrated in FIG. 9.

It also should be noted that if the feet on one side of cowl 320 are fixed, while the feet on the opposite side of cowl 320 are adjustable, the same type of curvature control can be provided. This can simplify the design of an externally-controlled adjustment mechanism.

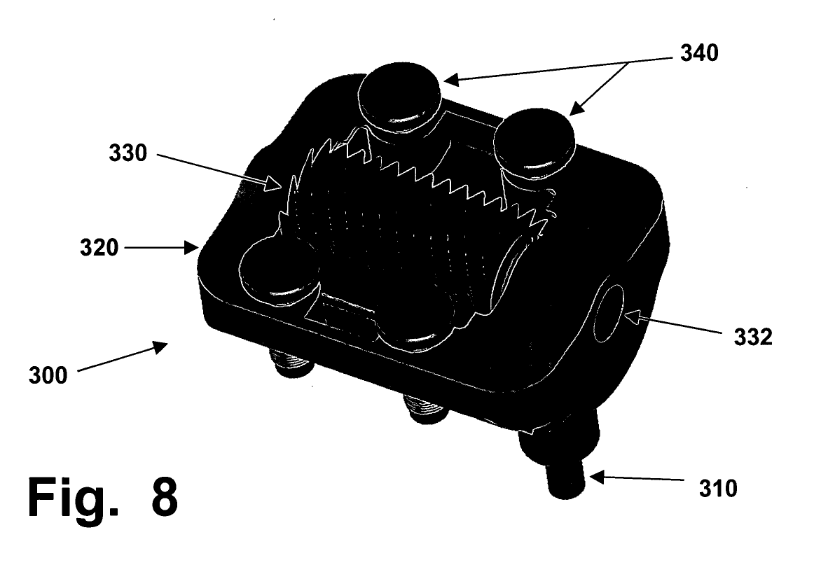

FIG. 8 provides a perspective view of the bottom side of femoral planing tool 300, showing burr 330 and shaft 332. If desired, a variety of burr surfaces can be made available on interchangeable tool heads. For example, some burrs can have rows of cutting edges or teeth, for rapid removal of cartilage and bone, while others can be provided with abrasive surfaces comparable to sandpaper, for final preparation of smooth surfaces. Similarly, some tools can contain cylindrical burrs, while other tools can contain concave, convex, or other semi-cylindrical burrs.

To indicate the general size of this type of tool, FIG. 9 depicts a femoral planing tool 300, pressed against a femoral runner 92 at the end of a femur bone 90. The bone depiction is stylized; during an operation, it will be surrounded by skin, ligaments, tendons, and other tissues.

FIG. 10 is a cutaway view of femoral planing tool 300, which illustrates how a gear surface at the lower tip 311 of driveshaft 310 can impart rotation to burr 330, via a radial gear surface 334 surrounding burr axle 332, on the end of burr 330. This drawing also illustrates curvilinear slot 316, which will allow curved plate 314 and driveshaft 310 to travel through a range or arc of motion, around the cylindrical top surface of cowl 320. Slot 316 also provides access to suction tube 318, an annular ring between sheath 312 and driveshaft 310, which allows removal of blood and particles entrained in saline solution that will be pumped through the joint during an operation, as described above.

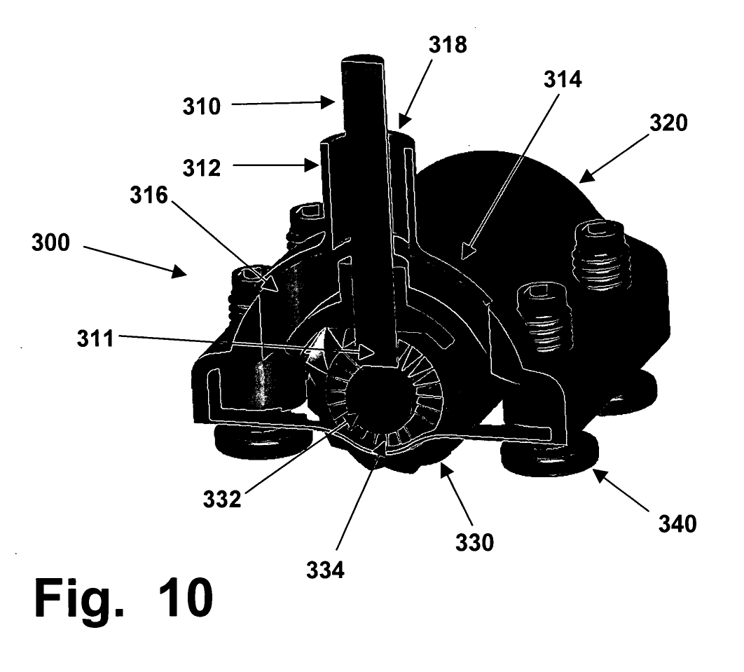

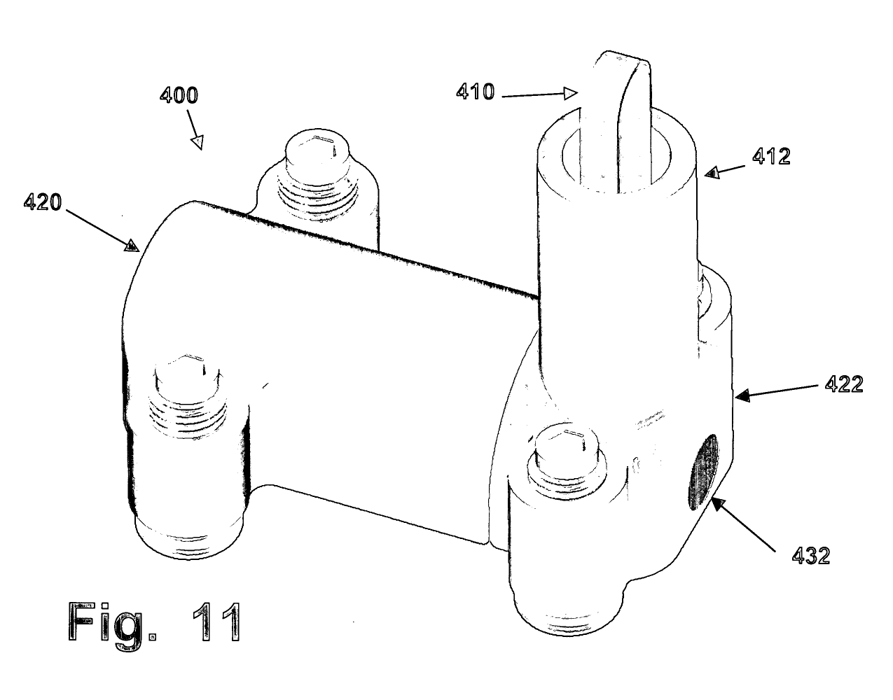

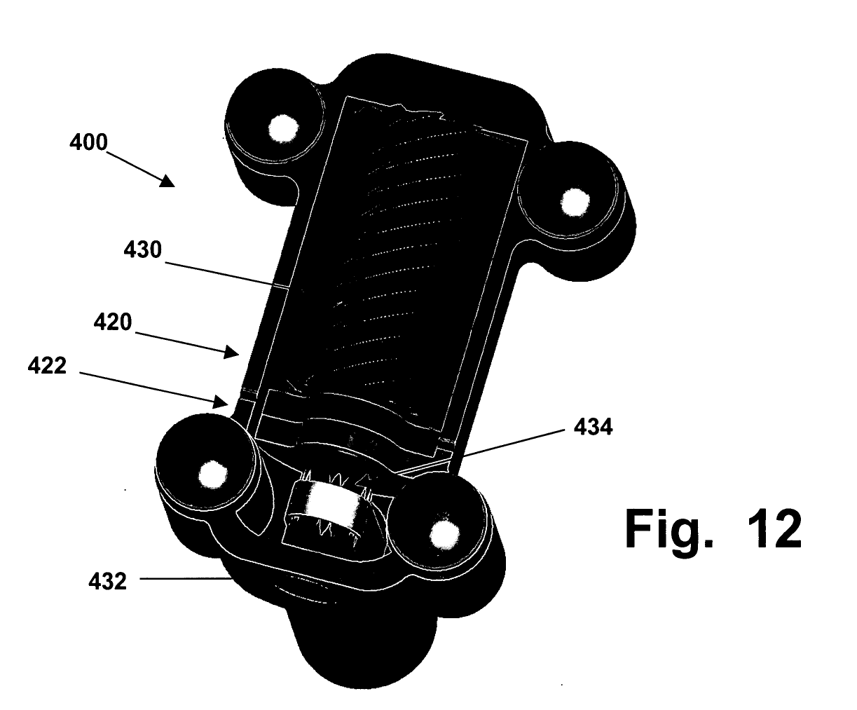

FIGS. 11 and 12 illustrate an alternate design for a femoral planing tool 400. As with planing tool 300, this design enables the sheath and handle component 410 to be perpendicular to axle 432 of burr 430. This design uses a thin and flexible drive belt 410, which will be operated in tension to drive the rotation of burr 430, by encircling and pulling against a non-smooth surface segment 434 of burr shaft 432, as shown in FIG. 12. Drive belt 410 passes through non-rotating sheath 412, which is mounted on a partial cowl component 422, which is affixed to main cowl component 420 in a rotatable manner.

If desired, drive belt 410 can travel to a powered drive mechanism that is a substantial distance away from the “operating head” of tool 400. Alternately, a chain-driven or similar mechanical drive mechanism with a powered and rotating non-smooth shaft can be inserted into the system, at a mid-point between the power-supplying drive unit, and the operating head. These types of potentially useful mechanisms, which have been extensively developed for various types of dental and orthopedic tools, can be adapted for use as described herein.

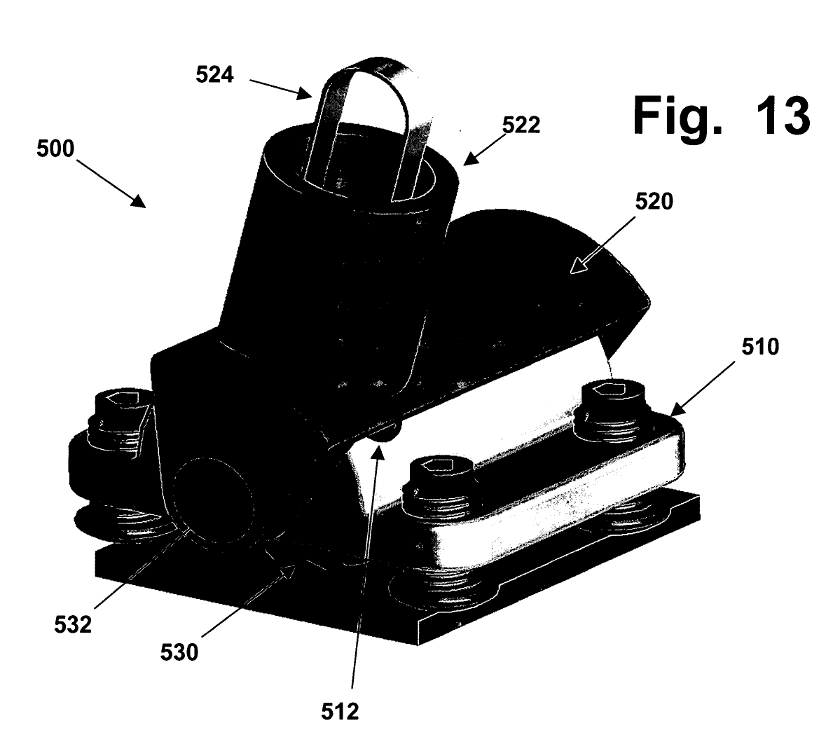

A third design for a femoral planing tool 500 is illustrated in FIGS. 13 and 14, which show a first cowl component 510 (which also can be called a lower cowl, foot cowl, bone cowl, or similar terms), and a second cowl component 520 (which also can be called an upper cowl, handle cowl, etc.). The two cowl components 510 and 520 can rotate through a substantial arc, with respect to each other. Upper cowl 520 has access tube 522 affixed to it, while lower cowl 510 has an accommodating slot 512 passing through it. Since access tube 522 will be constrained fairly tightly during use, by the requirement that it must pass through a relatively narrow tunnel that passes through the skin and various tendons and ligaments, during an arthroscopic procedure. Therefore, this design allows the “foot cowl” 510 to rotate through a substantial arc as it travels around the rounded surface of a femoral runner, while access tube 522 and upper cowl 520 remain at a constrained and relatively fixed angle.

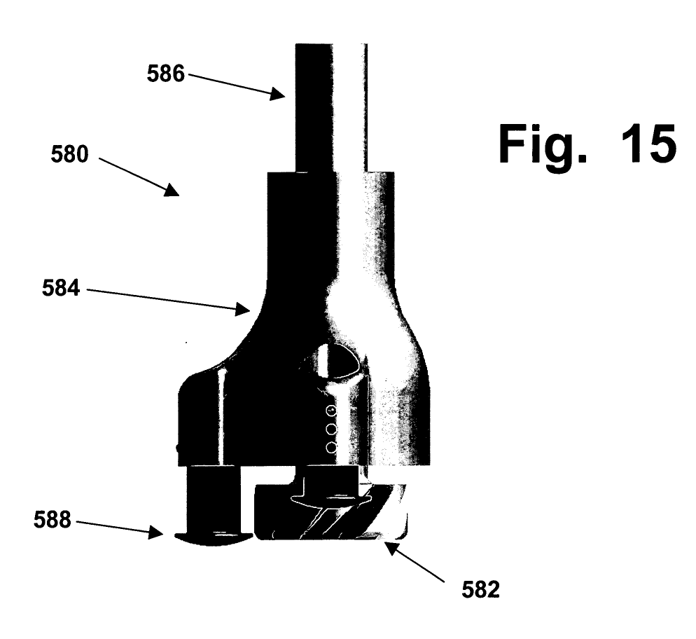

FIG. 15 depicts a side elevation view of another preferred design for a different type of bone-working tool 580, with an “end-mounted” burr 582 emerging from the end of cowl device 584 (shown truncated). Burr 582 is mounted at the end of rotating driveshaft 586 (also shown truncated). If burr 582 is provided with a sufficiently large diameter, it can be used as an end-mounted planing tool, and the working surface of the disc-shaped burr can be provided with a concave or convex shape. Alternately, if burr 582 has a narrower diameter, it can be used as a reaming tool, to create a circular hole with a fixed diameter, with a depth that can be controlled by adjusting feet 588. Such reamed holes can be useful, for example, for securing anchoring sleeves in a bone surface, to accommodate anchoring pegs affixed to an implant device, such as anchoring pegs 112 shown in FIG. 1.

Planing tools (either as described herein, or as developed by any other person or company) can be provided with or accompanied by and used with additional types of securing, guiding, or other systems. As just one example, prior to inserting a planing tool into a joint that is being prepared, a set of pins or screws having eyelets at the tops can be inserted into the bone, such as by using small drill holes that later will be enlarged and used to anchor the final implant. Strands of suture material can be placed through the eyelets, and secured to several points around the periphery of a planing tool. After the planing tool has been inserted into the joint, the strands can be tightened, and affixed to miniaturized winch-type devices that are operated and controlled by an electromechanical device that will remain outside the joint. The surgeon (or a computerized device) can operate the winches in ways that will use tension on the strands, passing through the peripherally-located eyelets inside the joint, to pull the planing tool in any desired direction, across the bone surface that is being prepared.

Other examples of guiding and control mechanisms, which can be used to control the movement of a planing tool across a bone surface, can use, for example, one or more relatively flat and thin curved metallic strips, which will have enough width to avoid flexure in an unwanted direction, while also using their curvature to press a tool against a bone surface with a desired degree of firmness.

Those are just two examples of guiding and control devices that would be suited for use with planing tools; others can be developed by those skilled in mechanical design.

### Tool for Preparing Controlled Grooves in Bone Surfaces

This section discloses a class of tools that will allow a groove (which can also be called a trench, furrow, rut, indent or indentation, or similar terms) to be created (or prepared, machined, dug, routed, etc.) in a bone surface, in a controllable and precise manner. If properly created, the groove can hold and help stabilize and reinforce an anchoring component of an implant device (such as a cartilage-replacing implant) that will be affixed to the bone. To simplify the description below, it is presumed that such an anchoring component normally will be in the form of a rim that effectively surrounds the entire periphery of the base (or other anchoring surface) of an implant, and that will effectively require a groove in the form of a complete circle, oval, ellipse, or similar shape. However, that is not essential, and the tools described herein can be used to create grooves with alternate shapes, whenever necessary, by using other types of templates, guides, or other control systems.

Presumably, this type of grooving operation will be carried out after a bone surface has been treated by a planing tool to create a relatively smooth surface. When the planed surface and the groove are both ready, an implant having an enlarged, strong, and moderately stiff rim (such as implant 100, illustrated in FIG. 1) can be positioned on the bone surface in a way that will cause the anchoring rim to settle securely into the groove. The implant will be anchored to the bone, by means such as pushing anchoring pegs (affixed to the rim of the implant) into anchoring sleeves that have been emplaced in the bone at several spaced locations. The “fit” of the anchoring rim in the groove will provide additional support, strength, and stability for the implant, which is especially important during the initial recovery period after surgery, when bony and/or scar tissue is just beginning to grow into a porous surface of the implant.

Accordingly, the tools disclosed herein must provide two different types of control over the grooves that will be formed by these tools. First, when viewed in cross-section, a groove must have a controlled and consistent depth, width, and shape, all of which should closely accommodate the cross-sectional dimensions (normally expressed as thickness, diameter, etc.) of an anchoring rim. Secondly, when viewed “from above”, the overall size and shape of the groove (i.e., its length, width, and exact shape) also must accommodate those additional corresponding dimensions of the anchoring rim.

Both sets of challenges can be met and overcome, by using a generally spherical grinding burr, inside a movable cowl mounted in a certain manner (described and illustrated herein) at the “working end” of the tool. A driveshaft will pass through a non-rotating sheath, which will function as part of a handle of the tool. The rapidly-spinning burr will extend a limited distance beyond the flat opening of the movable cowl, which will be shaped generally like a hemisphere, with a flat rounded opening. The cowl, and the end of the sheath, will have accommodating mounting components, which will enable the cowl to travel through a limited arc of motion while affixed to the end of the sheath.

As the cowl is rotated through its arc, at the end of the sheath, the spherical burr will continue to extend the same distance beyond the opening (or face) of the cowl. Therefore, so long as the opening of the cowl continues to be pressed firmly against the bone surface, while the tool is being moved across the bone surface, the spinning spherical burr will continue to grind a groove having a consistent depth, width, and semi-circular shape, regardless of the angle of the tool as it travels across the bone surface.

Therefore, when used with devices such as templates or guides (or a computerized motion-control system that will remains outside the joint), this tool will enable a surgeon to prepare a groove having consistent cross-sectional dimensions along its entire length, in a shape and size that will accommodate a pre-manufactured implant device having a strong anchoring rim.

Because of the design of this type of groove-forming tool, the groove that is formed will have a semi-circular (or “half-moon”) cross-section. This shape will hold the bottom half of an anchoring rim with a circular cross-section, in a way that will provide sufficient securing strength and reinforcement to meet the needs and goals of this invention, when combined with: (i) the anchoring pegs and receptacles that will also be used, and (ii) a porous bottom layer that promotes tissue ingrowth into the anchoring surface of the polymer layer that replaces hyaline cartilage.

Alternately, if desired, a semi-circular groove created by a grooving tool as described herein can be deepened, enlarged, or otherwise modified by one or more additional tools or procedures. As one example, a routing tool can be developed which will use a rounded steering or guiding mechanism that will fit into, and “ride in”, a semi-circular groove created by a grooving tool as described herein. The steering or guiding mechanism of this type of router tool can be followed closely by a spinning router bit which can have a desired shape, to create an enlarged trench having a corresponding cross-sectional shape.

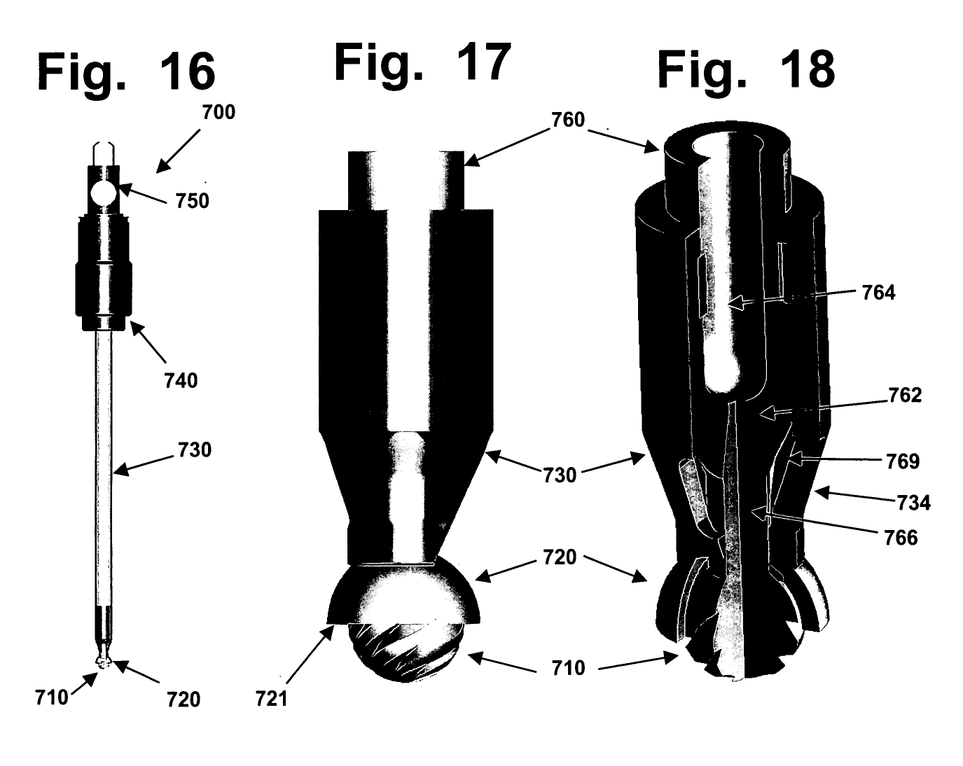

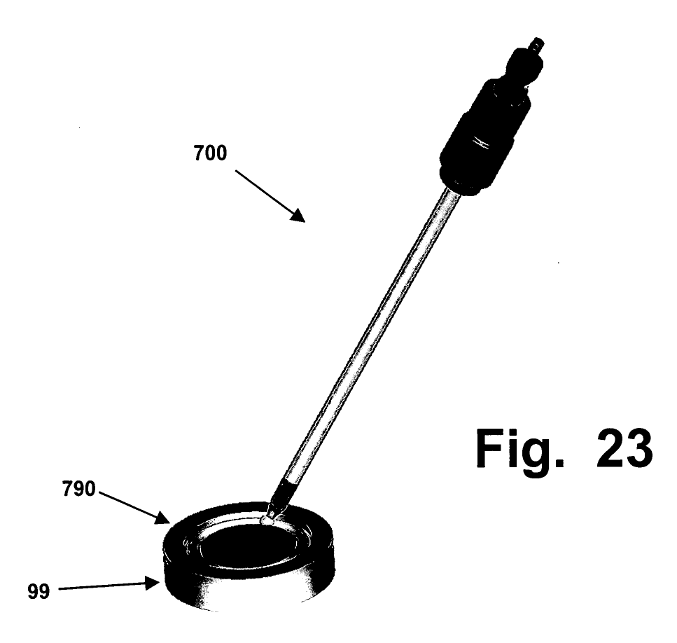

One embodiment of a grooving tool 700 that will create a semi-circular groove is illustrated in FIGS. 16-23. FIG. 16 shows tool 700, with its main components including a generally spherical grinding burr 710, a cowl 720, a hollow shaft 730, a non-rotating handle 740, and a drive interface 750. The handle 740 and drive interface 750 are designed to fit and lock (in a reversible manner) into a conventional commercially-available drive unit, as used by orthopedic surgeons for various other purposes.

Drive interface 750 is directly coupled to one end of a rotating driveshaft 760, shown (in truncated form) in FIGS. 17 and 18. The opposite (or “working”) end of driveshaft 760 is coupled to burr 710, via a transition zone or shoulder 762, described below.

FIGS. 17 and 18 show closer views of the “head” (or working head, working end, or similar terms) of tool 700, with the cowl “face” 721 (i.e., the planar opening of the cowl) oriented perpendicular to handle 730 and driveshaft 760. When the cowl is in that orientation, the burr will extent downward (or outward), beyond cowl face 721, a distance that is equal or nearly equal to the radius of the generally spherical burr. That radius distance will govern how deep the burr will cut into a bone, in a manner that digs and otherwise creates a groove in the bone, as cowl face 721 is pushed along the surface of a bone while being pressed firmly against the surface.

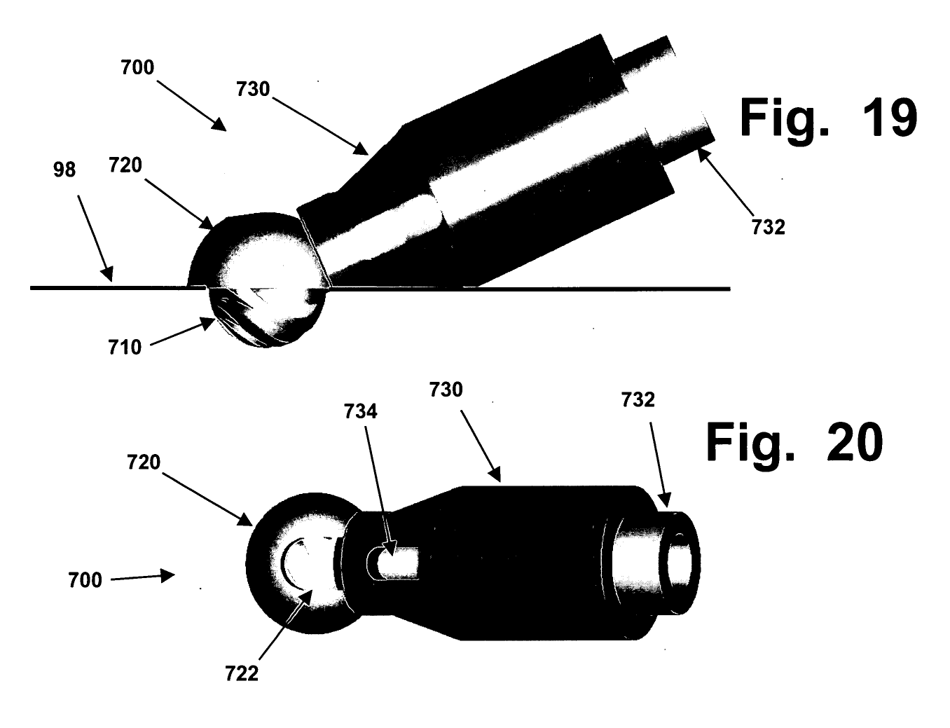

As indicated in FIG. 19, that same extension and/or depth of burr 710 (determined by the radius of the spherical burr) will be preserved even as cowl 720 travels through a rotational arc, while mounted at the end of sheath 730. That movement, by the cowl at the end of the sheath, can be referred to as rotation, since the cowl will move around an imaginary point located at the center of both the spherical burr 710, and the hemispherical cowl 720. Therefore, tool 700 will continue to create a groove having a uniform and consistent depth, equal to the radius of the spherical burr, so long as the cowl face 721 continues to be pressed firmly against a relatively smooth bone surface (represented by line 98, in FIG. 19) as the tool travels across the bone.

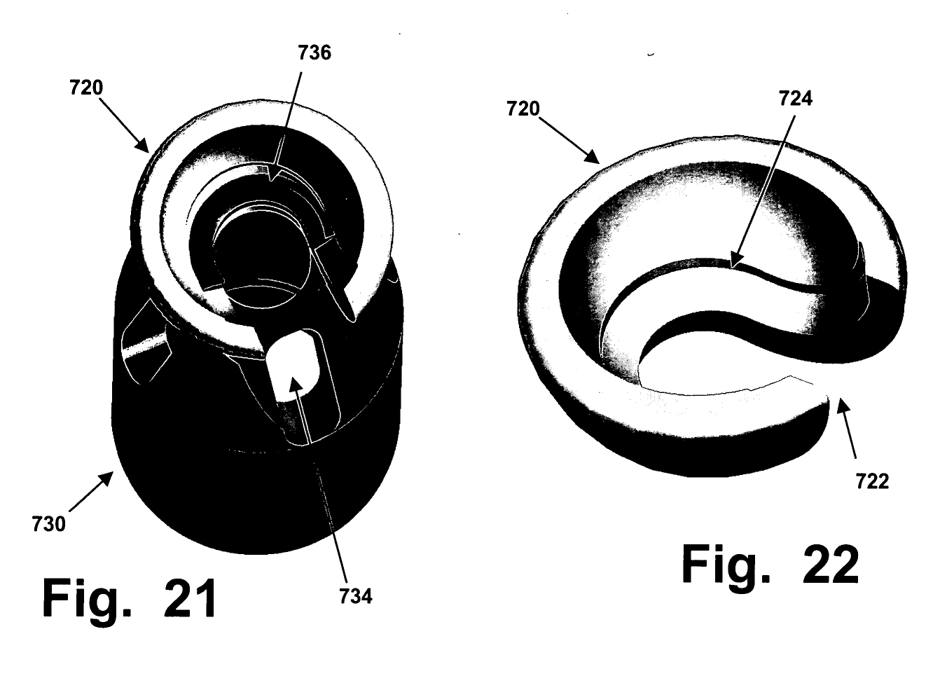

FIG. 20 depicts the head of tool 700 in an “overhead” view (assuming the tool is pointing downward at an angle, as shown in FIG. 19). This drawing shows a portion of cowl slot 722, which enables cowl 720 to travel through its rotational arc at the end of sheath 730. As shown in more detail in FIGS. 21 and 22, cowl slot 722 in cowl 720 is largely surrounded by a groove or depression 724, which receives and accommodates a rounded U-shaped lip 736 affixed to the bottom tip of sheath 730.

FIGS. 18, 20, and 21 also show one or more suction slots 734, passing through the outer wall of sheath 730. As indicated by the cutaway view in FIG. 18, driveshaft 760 will have a transition zone 762 which can be called a shoulder or similar terms. At that location, driveshaft 760 changes from a hollow tube with a center passageway (or suction conduit) 764 above shoulder 762, to a narrow solid “neck” 766 below the shoulder.

The size and shape of shoulder 762 is designed to create a small and generally conical gap 769, between driveshaft 760 and sheath 730. One or more slotted openings will pass through the shoulder 762, in a manner that will allow saline solution with entrained blood and particles to be suctioned through the sheath slots 734 and the conical gap 769, into the suction conduit 764 located in the center of driveshaft 760. The slotted openings passing through shoulder 762 of driveshaft 760 preferably should be angled, in a manner that will create a suction force when the driveshaft rotates rapidly. That suctioning force will promote the clearance of saline, blood, and particles out of the operating zone.

FIG. 22 depicts grooving tool 700 being used to create a groove in a hard surface 99 (such as a bone surface). The overall size and shape of the groove are controlled by temporarily anchoring a template or guide device 790 (comprising a hard flat ring of material) on top of the bone or other material 99.

Alternately or additionally, various other means can be used to control the size and shape of a planed and/or grooved bone surface. As an example, various types of computer-controlled robotic arms are used to perform high-precision machining operations, often with tolerances measured in thousandths of an inch or even less. During an operation as described herein, a harness-type device comparable to a large knee brace can be tightly secured to both the thigh and calf of a patient, and one or more pins, pads, or other devices can be driven into or pressed against one or more bones, to provide exact and stable positioning for a robotic arm that will be used to carry out planing and/or groove-forming procedures on a bone surface inside a knee or other joint. This type of machining approach can allow a robotic device in a surgeon's office to rapidly create a planed surface and a groove having a precise size and shape, in a tibial plateau or other bone. The machinery (including the computer interfaces and programming) for using robotic arms for these types of machining operations has been extensively developed, and can be adapted for use as described herein by those skilled in that line of art.

Thus, there has been shown and described a new type of surgical implant for replacing meniscal or labral cartilage in knees and other joints, along with methods and tools for strongly anchoring such implants to both hard bone and soft tissue. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A surgical implant for replacing at least one segment of meniscal cartilage in a mammalian knee, comprising:
a. at least one anchoring rim component, which is sized to be affixed to a tibial bone, and which is positioned around or adjacent to at least one outer edge of an anchoring base of said surgical implant;
b. at least one flexible polymer component, sized to replace a segment of meniscal cartilage, and affixed to said anchoring component; and,
c. at least one flexible securing component affixed to said flexible polymer component, and suited for fixation to soft tissue in a knee joint.

2. A surgical implant of claim 1, wherein said anchoring component, when affixed to a bone, and said flexible securing component, when affixed to soft tissue, will establish anchoring connections along or adjacent to two spaced and opposing edges of said flexible polymer component.

3. A surgical implant of claim 1, wherein said anchoring rim component comprises a continuous rim that encloses an anchoring base of said surgical implant.

4. A surgical implant of claim 3, wherein said anchoring rim comprises a generally annular ring with an open slot, made of a shape-memory material and having dimensions that allow said annular ring to securely grip and hold a flexible polymer insert.

5. A surgical implant of claim 3, wherein said anchoring rim comprises a metallic ring embedded within a polymer ring.

6. A surgical implant of claim 3, wherein a plurality of anchoring pegs are affixed to said anchoring component.

7. A surgical implant of claim 6, wherein:
a. said flexible polymer component has an arc-like semi-circular shape, with arcuate tips at two opposed ends of said flexible polymer component; and,
b. flexible securing means emerge from both of said arcuate tips, in a manner which provides a surgeon with means for anchoring each of said arcuate tips to an attachment location in a joint that is being repaired.

8. A surgical implant of claim 1, which also comprises a second flexible polymer component which is sized to replace a segment of hyaline cartilage in a joint being repaired.

9. A surgical implant of claim 8, wherein said second flexible polymer component is reinforced by a flexible fibrous reinforcing layer that is affixed to said anchoring component.

10. A surgical implant of claim 8, wherein said second flexible polymer component has a porous anchoring surface which will promote tissue ingrowth after surgical implantation.

11. A surgical implant for replacing hyaline cartilage and meniscal or labral cartilage in a mammalian joint, comprising:
a. at least one first polymer component made of a nonrigid hydrophilic polymer which is sized to replace a segment of hyaline cartilage, wherein said first polymer component has both (i) a porous anchoring surface which will promote tissue ingrowth into said anchoring surface after surgical implantation, and (ii) a smooth and wettable articulating surface which will replace an articulating surface of natural hyaline cartilage;
b. at least one second polymer component made of a nonrigid hydrophilic polymer, which is sized to replace a segment of meniscal or labral cartilage in a mammalian joint, wherein said second polymer component is provided with at least one smooth and wettable articulating surface;
c. at least one anchoring component, sized to be affixed to a bone; and,
d. means for affixing said first and second polymer components to said anchoring component.

12. A surgical implant of claim 11, wherein a slidable interface separates said first polymer component from said second polymer component, in a manner that will allow synovial fluid to enter and lubricate said slidable interface, after surgical implantation of said surgical implant.

13. A surgical implant of claim 11, wherein said first polymer component and said second polymer component are molded together to form an integral flexible polymer component, with no slidable interface separating said first polymer component from said second polymer component.

14. A surgical implant of claim 11, wherein said first polymer component and said second polymer component are reinforced by flexible fibrous materials which are (i) partially embedded within each of said first and second polymer components, and (ii) affixed to an anchoring component that is sized to be affixed to a hard bone.

15. A surgical implant of claim 11, wherein said anchoring component comprises a continuous rim around a periphery of an anchoring base of said surgical implant.

16. A method for repairing a mammalian joint having at least one hyaline cartilage surface and at least one meniscal or labral cartilage surface, comprising the following steps:
a. creating a prepared subchondral bone surface that will receive and support a prosthetic implant, by preparing a smooth surface on at least a portion of a bone;
b. anchoring a joint resurfacing implant to said prepared subchondral bone surface, wherein said joint resurfacing implant comprises: (i) at least one anchoring component sized to be affixed to subchondral bone; (ii) at least one first polymer component designed to replace a hyaline cartilage segment and which is affixed to said anchoring component; and, (iii) at least one second polymer component sized to replace a meniscal or labral cartilage segment.

17. The method of claim 16, wherein said second flexible polymer component has at least one additional flexible securing means that is accessible along at least one peripheral edge of said second flexible polymer component, and wherein said flexible securing means is suited to be sutured or stapled to soft tissue in or around the knee joint.

18. The method of claim 16, wherein said joint resurfacing implant also comprises at least one porous surface which will promote tissue ingrowth into said porous surface after surgical implantation.

19. The method of claim 16, wherein at least one anchoring component comprises a continuous rim around a periphery of an anchoring base of said joint resurfacing implant.

20. A method for resurfacing a joint surface having a hyaline cartilage surface, comprising:
a. smoothing a joint surface to a subchondral bone layer;
b. forming at least one cavity in said subchondral bone layer, underlying a peripheral portion of said subchondral bone layer; and,
c. anchoring a joint resurfacing implant to said subchondral bone layer, with at least one anchoring portion of said joint resurfacing implant extending into said cavity.

21. The method of claim 20, wherein at least one anchoring portion of said joint resurfacing implant comprises a continuous rim around a periphery of said joint resurfacing implant.

22. The method of claim 21, wherein at least one cavity-forming step comprises creation of a groove that is sized to accommodate a continuous rim around a periphery of said joint resurfacing implant.

23. The method of claim 20, wherein said joint resurfacing implant comprises a plurality of anchoring pegs that are spaced around a periphery of said joint resurfacing implant.

24. The method of claim 20, wherein said joint resurfacing implant also comprises a polymer component that is sized to replace a segment of meniscal or labral tissue in a joint that is being resurfaced.

25. The method of claims 24, wherein said joint comprises a knee joint, and wherein said joint resurfacing implant comprises a polymer component that is sized to replace a segment of meniscal cartilage, and wherein at least one flexible securing means that is affixed to said polymer component is affixed to soft tissue within said knee joint.
